SEQUENCE LISTING (22 sequences)

SEQ ID NO: 1 - DNA, 20 bp, Artificial sequence
  Primer
gcagtacagccccaaaatgg

SEQ ID NO: 2 - DNA, 21 bp, Artificial sequence
  Primer
ggtccttttcaccagcaagct

SEQ ID NO: 3 - DNA, 29 bp, Artificial sequence
  Primer
aacaagactaccctttactgaaattacca

SEQ ID NO: 4 - DNA, 22 bp, Artificial sequence
  Primer
aatggcatctctgtgtcaacca

SEQ ID NO: 5 - DNA, 18 bp, Artificial sequence
  Primer
gcatggtgccttcgctga

SEQ ID NO: 6 - DNA, 22 bp, Artificial sequence
  Primer
tggcatctctgtgtcaaccatg

SEQ ID NO: 7 - DNA, 20 bp, Artificial sequence
  Primer
ccagtgcccaggaagaagac

SEQ ID NO: 8 - DNA, 20 bp, Artificial sequence
  Primer
cgaacgtcatctggcacttg

SEQ ID NO: 9 - DNA, 21 bp, Artificial sequence
  Primer
cagcaatgggatacggacaga

SEQ ID NO: 10 - DNA, 21 bp, Artificial sequence
  Primer
gagtcgatgaggcgcacatag

SEQ ID NO: 11 - DNA, 21 bp, Artificial sequence
  Primer
gctagggttggagaggaatgg

SEQ ID NO: 12 - DNA, 24 bp, Artificial sequence
  Primer
aaaagtaaccgggatgaatgtctg

SEQ ID NO: 13 - DNA, 21 bp, Artificial sequence
  Primer
gagagagcccaaagggacaaa

SEQ ID NO: 14 - DNA, 24 bp, Artificial sequence
  Primer
tggcagtccttgagtctcacatag

SEQ ID NO: 15 - DNA, 20 bp, Artificial sequence
  Primer
ccgtacttccggacgacttc

SEQ ID NO: 16 - DNA, 24 bp, Artificial sequence
  Primer
gactgtctccaggtgtcatattgg

SEQ ID NO: 17 - DNA, 23 bp, Artificial sequence
  Primer
tcgtgttctctcgaggtctcact

SEQ ID NO: 18 - DNA, 21 bp, Artificial sequence
  Primer
tgggtcgagtatacggtccaa

SEQ ID NO: 19 - DNA, 22 bp, Artificial sequence
  Primer
gactgacgcaaacacaggtgaa

SEQ ID NO: 20 - DNA, 26 bp, Artificial sequence
  Primer
gatcttggatcttttgttctgaaacc

SEQ ID NO: 21 - DNA, 22 bp, Artificial sequence
  Primer
cacgtactctcctcccctcaat

SEQ ID NO: 22 - DNA, 20 bp, Artificial sequence
  Primer
aactgcacagggcacgtctt

(19) United States
(12) Patent Application Publication
(10) Pub. No.: US 2014/0377832 A1
(43) Pub. Date: Dec. 25, 2014

(54) INDUCTION OF DEDIFFERENTIATION OF MESENCHYMAL STROMAL CELLS

(71) Applicant: YEDA RESEARCH AND DEVELOPMENT CO. LTD., Rehovot (IL)

(72) Inventors: Dov Zipori, Rehovot (IL); Ofer Shoshani, Rehovot (IL)

(21) Appl. No.: 14/371,640

(22) PCT Filed: Jan. 14, 2013
(86) PCT No.: PCT/IL13/50035

§ 371 Date: Jul. 10, 2014

Related U.S. Application Data
(60) Provisional application No. 61/586,791, filed on Jan. 15, 2012.

(51) Int. Cl. C12N 5/074; C12N 13/00

(52) U.S. Cl. CPC C12N 5/0696; C12N 13/00; C12N 2506/21; C12N 2500/02
USPC 435/173.1; 435/377; 435/325; 435/366

(57) ABSTRACT

The invention relates to induction of reprogramming of somatic cells, by methods which require mild growth conditions. Disclosed are methods of inducing dedifferentiation of mesenchymal stromal cell (MSC), by seeding or incubating mesenchymal stromal cells (MSCs) at low density, and without introduction or expression of exogenous genes in the cells.

### FIELD OF THE INVENTION

The invention relates to the field of cell reprogramming (dedifferentiation) under specific induction conditions, without the introduction or expression of exogenous genes.

### BACKGROUND OF THE INVENTION

The bone marrow is a unique environment, harboring many cell types, which are arranged in an elaborate tri-dimensional structure. Originally, this compartment was found to be the source of hemopoietic cells in adult life. However, other cellular constituents of the bone marrow were disregarded until experiments showing that fibroblastic cells derived from the bone marrow have bone-forming capacity, and more importantly, are able to create an ectopic bone marrow environment in vivo. The cells belonging to this fibroblastic population were given many different designations including osteoprogenitor cells, fibroblastoid cells, stromal cells, colony forming unit-fibroblasts (CFU-F), mesenchymal cells and finally, mesenchymal stem cells/multipotent stromal cells/mesenchymal stromal cells (MSCs). The exact in vivo origin of MSCs is not certain and their definition is based mainly on their in vitro growth properties and capacity to differentiate. The derivation of continuously growing stromal cell populations from the bone marrow revealed the heterogeneity of this population. Among the many different cell types discovered were fibroblasts, adipocytes, endothelial cells, osteogenic cells, and more, all with distinct morphologies. Clonal populations of such stromal cells were shown to have the potential to differentiate into three cell types: adipocytes (fat cells), osteocytes (bone cells) and chondrocytes (cartilage cells).

Initially, stromal cells were considered to be structural entities, scaffolding the compartment in which hemopoiesis occurs. This underestimation was slowly being abandoned, as more functions of these cells were discovered. Tissue culture work revealed that these cells are capable of creating conditions which allow long-term maintenance of hemopoiesis. It was also demonstrated that MSCs possess immuno-modulatory functions, such as T cell suppression. In addition, MSCs carry different immune system related molecules such as toll-like receptors (TLRs), T cell receptors (TCRs) and B cell receptor components.

MSC are not known to possess unique surface markers, which could make it possible to identify them in vivo. A plethora of molecules have been suggested as possible MSC markers (reviewed in ref 1). Other studies suggest a CD146 positive phenotype of human MSCs, which were identified in vivo as adventitial reticular cells (ARC) or otherwise pericytes. In the mouse, similar cells were identified in vivo as being PDGFRα⁺Sca-1⁺ cells. The standard method for deriving MSCs is by negative selection (i.e., removing other cells, such as CD11b macrophages). However, certainty regarding the success in derivation of MSCs is reached only after these cells are grown in culture and tested for their capacity to differentiate into at least three cell types: adipocytes, osteocytes and chondrocytes. MSCs are not unique to the bone marrow and actually exist in other body compartments as well, such as adipose tissues, ears, cord blood, placenta and many more. They therefore represent a multipotent progenitor population which is tissue non-specific, exhibiting body wide distribution.

Early works in plant biology (2), as well as on primitive organisms such as Drosophila (3) have taught us that cells have greater potential than previously expected. It appears that under some circumstances, terminally differentiated mature cells have the ability to shift into a more permissive state, which enables them to act as stem cells and to regenerate the damaged tissue. Until recently, such cellular plasticity was thought to be absent in mammals. The classical example is the view that differentiation of the hematopoietic stem cell (HSC) follows a strict hierarchical one-way path enabling differentiation ending in terminal commitment. This notion has been challenged in recent years, as accumulating evidence show that plasticity is a possible feature in the arsenal of cellular behaviors in mammalian tissues (reviewed in 1 and 4).

Work on mouse spermatogonia showed an example of in vivo dedifferentiation. Cell lineage tracing is relatively convenient in the testis, as the cells form orderly syncytial cysts, which reflect the history of cell division. It appears that under steady state conditions, the spermatogonial cells exhibit primarily a functional hierarchy in which the GFRα1⁺ Aₛ cells act to maintain cell types and amounts. This observation has a rare exception, as some NGN3⁺ revert back and become GFRα1⁺ cells through cyst fragmentation events. This rare phenomenon becomes widespread upon administration of busulfan, a specific drug, which is toxic to spermatogonia. Tissue regeneration is achieved primarily by cell reversion from more committed NGN3⁺ back into GFRα1⁺ cells. The contradictory paths shown in steady state and regeneration processes emphasize the ability of the tissue to adjust to different needs. Further evidence for the ability of cells to reprogram comes from the work on pancreatic α- and β-cells. Following an almost complete ablation of β-cells (>99%), bihormonal cells which secrete insulin and glucagon arise from the α-cell population. Some of these dedifferentiated cells eventually lost their glucagon secretion ability, thus completing the trans-differentiation process from α- to β-cells. Apparently, when performing less critical ablation of β-cells (50%), induction of cell reprogramming decreases, and the spared β-cells seem to play a more important role in regeneration of the damage.

The underlying mechanisms by which cells dedifferentiate are widely unknown. There are many possible routes to be taken once cells set their course to dedifferentiate. The stem state theory postulates that cells are able to enter a permissive state in which they express most, if not all of their genome, at very low levels. Once the cell embarks on a differentiation path, it narrows its gene expression profile specifically, according to the functions it acquires. Work on embryonic stem cells (ESCs) revealed a global transcription pattern, which diminishes along the differentiation course of the cells. This data indicates that if a cell selects the reverse path, i.e, dedifferentiation, it should loosen its chromatin and enable such global gene expression. Indeed, it was shown that genome organization through histone modifications and DNA methylations are responsible for heterochromatin formation and promoter repression, respectively, which prevents gene expression. In addition, it appears that heterochromatin formation itself does not prevent cell reprogramming, rather, the de novo methylation of the DNA locks cells in a non reprogrammable state. Thus, epigenetic changes such as histone modifications are reversible, and allow a bi-directional course for cells to differentiate or dedifferentiate according to specific conditions. In this context, a possible role for Chd1, a chromatin remodeling factor, was suggested as a regulator of open chromatin and reprogramming of cells. Once DNA is methylated, however, the cells need to demethylate it in order to reprogram. Demethylation is considered to be a tougher barrier for cell reprogramming, however it may still be reverted. The activation-induced cytidine deaminase (AID) was shown to be able to bind silent promoters and reactivate them.

There are several ways to artificially reprogram cells in culture. One possible way is by transferring the nucleus of a mature cell into an oocyte. It appears that the oocyte serves as permissive environment upon the introduced nucleus, thus allowing it to be reprogrammed (6). The exact mechanism by which the nucleus is reset is still unknown, and the involvement of molecules, which act to relax the chromatin is suggested. With the introduction of induced pluripotent stem cells (iPS cells), it was shown that overexpression of the genes Oct3/4, Sox2, c-Myc, and Klf4, is sufficient to turn mature fibroblasts into pluripotent cells. (7). For example, US 2009/0068742, entitled “Nuclear Reprogramming Factor” is directed to a nuclear reprogramming factor for a somatic cell, which comprises a gene product of each of the following three kinds of genes: an Oct family gene, a Klf family gene, and a Myc family gene, as a means for inducing reprogramming of a differentiated cell to conveniently and highly reproducibly establish an induced pluripotent stem cell having pluripotency and growth ability similar to those of ES cells without using embryos or ES cell. Another study demonstrated that the iPS cells have a remodeled epigenome, which resembles that of ESCs. Furthermore, X chromosomes are reactivated upon reprogramming, and upon differentiation are randomly inactivated (8). US 2008/2033610, entitled “Somatic Cell Reprogramming” relates to methods for reprogramming a somatic cell to pluripotency by administering into the somatic cell at least on or a plurality of potency-determining factors. Other studies revealed that adult unipotent germline cells can be induced to become pluripotent, without the use of gene delivery (10). The cells require mouse embryonic fibroblast (MEF) feeder cells for expansion as well as for reprogramming, but, apparently, lower density seeding and longer culture time yield higher percentages of induced cells. It is possible that these conditions simulate the niche required for the dedifferentiation process to take place. Actually, spermatogonial cells in the fly dedifferentiate by relocating in proximity to the hub, which supplies the correct microenvironment for the process (11). An additional study demonstrated the possibility of stem and progenitor spermatogonia to transdifferentiate in vivo into tissues of all germ layers (10).

Nevertheless, there remains an unmet need in the art for simplified methods for inducing dedifferentiation of somatic (non germline cells) that use mild conditions and avoid the use of transfection, infection and/or overexpression of exogenous genes. In particular, there is a need in the art for simplified methods for inducing dedifferentiation of mesenchymal stromal cells by using mild and specific growth conditions, and avoiding the use of transfecting, infecting or overexpressing exogenous gene(s) in the cells.

### SUMMARY OF THE INVENTION

The present invention is directed to novel methods for the induction of dedifferentiation of somatic cells, such as, mesenchymal stromal cells (MSCs), wherein the methods utilize mild conditions and do not involve overexpression of exogenous genes to induce such dedifferentiation of the cells.

The present invention further provides methods for the reprogramming of somatic cells by inducing the cells to re differentiate to other desired cell types, wherein the methods do not include introduction or overexpression of exogenous genes in the cells.

According to some embodiments, the present invention is based on the unexpected and surprising finding of methods of inducing cell de-differentiation, in particular of mesenchymal stromal cells, wherein the methods use mild conditions for the induction and do not involve the use of transfection and/or infection and/or overexpression of exogenous genes in these cells. As exemplified hereinbelow, the methods of the present invention enable de-differentiation of mesenchymal stromal cells, whereby the cells gain differentiation potential. In some exemplary embodiments, mesenchymal cells that lack differentiation potential altogether (i.e., impotent cells also referred to herein as nullipotent cells) gain unipotentiality. In some exemplary embodiments, uni-potent cells gain bi-potency or multipotency. In some exemplary embodiments, bi-potent cells gain multipotency.

According to some exemplary embodiments, and as further demonstrated herein below, using the methods of the invention, bi-potent mesenchymal cells that were able to differentiate only into osteocytes and chondrocytes, are de-differentiated and gain re-differentiation potential and become multipotent as they acquire epithelial and endothelial morphologies as well as adipogenesis capability. In other exemplary embodiments, cells that lack chodrogenic potential, regain it by using the methods of the present invention.

In some embodiments, the methods of inducing de-differentiation of mesenchymal stem cells comprise incubating/growing the cells at low density (for example, at a density of about 2000 cells/0.3 cm² or less), and may optionally further include varying one or more physical and/or chemical environmental conditions in which the cells are grown. For example, the physical and/or chemical environmental conditions of the cells, may be selected from, but not limited to: growth medium, temperature, CO₂ concentration, O₂ concentration, pH, pressure, humidity, substrate, type of plate in which the cells are grown, irradiation, and the like, or combinations thereof. In some embodiments, the induction of de-differentiation involves various intracellular cell-signaling pathways. In some embodiments, the methods for inducing de-differentiation of mesenchymal stem cells exclude the introduction or expression of exogenous genes in the mesenchymal stromal cells or any other genetic manipulation/modification of the cells.

According to some embodiments, there is thus provided a method for inducing de-differentiation of mesenchymal stromal cell (MSC), the method comprising seeding or incubating mesenchymal stromal cell (MSC) at low density of less than about 2000 cells/0.3 cm²; thereby inducing de-differentiation of the mesenchymal stromal cell (MSC). In some embodiments, an exogenous gene is not expressed or introduced into the mesenchymal stromal cell (MSC). In some embodiments, a single cell derived mesenchymal stromal cell colony is obtained.

In some embodiments, the de-differentiation process is from an un-differentiated mesenchymal stem cell (impotent cell) to: a uni-potent stem cell, a bi-potent stem cell, a tri-potent stem cell or a multi-potent stem cell. In other embodiments, the de-differentiation process is from a uni-potent mesenchymal stem cell to: a bi-potent stem cell, a tri-potent stem cell or a multi-potent stem cell. In additional embodies, the de-differentiation process is from a bi-potent mesenchymal stromal stem cell to a tri potent stem cell or a multi-potent stem cell.

According to further embodiments, the mesenchymal stromal cell (MSC) is de-differentiated to a cell capable of differentiating to: an osteogenic cell type, an adipogenic cell type, and/or a chondrogenic cell type.

In some embodiments, the low density is less than about 1000 cells/0.3 cm². In some embodiments, the low density is less than about 500 cells/0.3 cm². In some embodiments, the low density is less than about 100 cells/0.3 cm².

According to some embodiments, the method may further include a step of changing one or more growth conditions of the MSC. In some embodiments, the growth conditions may be selected from, but not limited to: growth media, O₂ concentration, CO₂ concentration, pressure, humidity, pH, temperature, type of substrate, or combinations thereof. According to further embodiments, the method may further include a step of irradiating the cells with one or more types of irradiation (such as, for example X-ray, UV), at various intensities.

According to additional embodiments, the mesenchymal stromal cell (MSC) may be obtained from human or animal origin, such as, for example, but not limited to: murine, canine, poultry, cattle, farm animals, cats, primates (chimps and other monkeys), birds, and the like.

In further embodiments, the mesenchymal stromal cell may be derived from, but not limited to: bone marrow, adipose tissue, spleen tissue, intestine, liver tissue, muscle tissue, brain, skin, ear, bone/cartilage tissues, dental tissue, embryonic tissue, cord blood, placenta, heart, nervous system, spinal cord and the like, or combinations thereof.

In additional embodiments, the dedifferentiated mesenchymal stromal cell (MSC) is capable of being introduced to a human or animal.

According to some embodiments, there is further provided a dedifferentiated mesenchymal stromal cell obtained by a method comprising a step of seeding or incubating a mesenchymal stromal cell (MSC) at low density of less than about 1000 cells/0.3 cm²; and wherein an exogenous gene is not expressed in or introduced into the mesenchymal stromal cell (MSC).

Other objects, features and advantages of the present invention will become clear from the following description and drawings.

### BRIEF DESCRIPTION OF THE DRAWINGS

FIGS. 1A-C show Bone marrow derived stromal cells have variable differentiation potentials. FIG. 1A A table showing 12 independent derivations that were examined for their differentiation into adipocytes, osteocytes and chondrocytes in induction media. FIG. 1B shows micrographs of cells stained with Oil red O (adipocytes), alizarin red (osteocytes) and Alcian blue (chondrocytes). Insets show typical chondrocytic morphology. FIG. 1C shows Fluorescence Activated Cell Sorting (FACS) analysis of surface marker analysis with antibodies for CD45 (hematopoietic cell marker), CD11b (macrophage marker) and Sca-1 (presumed MSC marker). line: antibody (Ab), Dark line: Isotype control (Ct).

FIGS. 2A-G—pictograms of spontaneous multipotent differentiation of MSCs after low density seeding. FIG. 2A MSC OC seeded at normal density (1:4 passage), was allowed to reach confluence and showed homogenous morphology. FIGS. 2B-F show MSC OC after two rounds of low density seeding (first 15,000 cells in 100 mm dish reached confluence, collected, reseeded at 2,500 cells in 100 mm dish and allowed to reach confluence). Scale bar −200 μm. Fig G (G.1-left panel and G.2-right panel) MSC OD after low density seeding (2,500 cells seeded in 100 mm dish) show spontaneous adipocytic differentiation.

FIGS. 3A-B—MSC clones lose and gain differentiation potentials. FIG. 3A—Tables summarizing comparison of 23 MSC OA clones (top table) and 25 MSC OC clones (bottom table) at early (passage 2) and late (passage 12) passages. Diagonal line represents clones with unchanged potentials during passaging. Below the diagonal line are cells which lost potentials during passaging, and above are clones which gained potentials. O—Osteogenesis, A—Adipogenesis, C—Chondorgenesis. FIG. 3B—pictograms of two MSC OC clones negative for chondrogenic differentiation at an early passage, were found positive with alcian blue staining at the later passage.

FIG. 4—A diagram showing lineage tree of MSC OC clone 4 (MSC OC.4). Early and late passages of MSC OC.4 were re-cloned by 0.2 cell seeding into 96-well plates. This process of cellular cloning was repeated with selected sub-clones until reaching quaternary clones. All clones were subjected to tri-lineage differentiation assays (Black—osteogenesis, light gray—adipogenesis, Dark gray—chondrogenesis). * Quaternary clones were not examined for chondrogenic differentiation. Circled clones are selected clones which show the loss and gain of adipogenic potential. Horizontal line represents passaging in culture (PD—population doublings).

FIGS. 5A-B—Selected clones in MSC OC.4 lineage lose and gain adipogenic potential. FIG. 5A—pictograms of MSC OC cells population and its descendent clones that were assayed for adipogenic differentiation and stained with Oil red O (pictures are representatives of three independent repeats). FIG. 5B—pictograms of MSC OC and MSC OC.4L.1.4 cells that were seeded at low density (100,000 cells in 100 mm plate) and were stained for OCT4 one day after seeding. Embryonic stem cells (ESC) served as positive controls and primary splenocytes as negative controls. MSC OC showed higher fluorescence than OC.4L.1.4 (inset shows dim fluorescence in cells).

FIG. 6—Ploidy of MSC OC and its clones is stable as shown by FACS analysis. Top left: MSC OC (solid (originally blue) line) was compared to primary diploid spleen cells (dashed (originally red) line), and was found to be near tetraploid by FACS analysis. Clones are represented by a dashed (originally red) line compared to MSC OC population (solid (originally blue)). With the exception of MSC OC.4L.2 (which has an octaploid sub-population), all clones align with MSC OC ploidy with high similarity.

FIG. 7—Acquisition of adipogenic potential is accompanied by changes in adipogenic gene expression. MSC OC and its derivative clones, before and after adipogenic induction in culture (control/induced), were subjected to real-time PCR analysis with four different genes (Znf423, PPARγ-1, Ebf1 and PPARγ-2). The highly adipogenic MSC OA served as a control. Levels of gene expression were evaluated relative to HPRT housekeeping gene. The results shown in FIG. 7 are of bar graphs plotted on a logarithmic scale (except for Ebf1), showing the relative mRNA level (relative to HPRT) of Znf423, PPARγ-1 (PPARG-1), Ebf1 and PPARγ-2 (PPARG-1) in various MSC cells and derivatives clones thereof. Experiments were repeated two independent times with two repeats each time.

FIG. 8—A Schematic alignment of differentially expressed genes and their chromosomal locations. Four chromosomes had significantly more upregulated genes in MSC OC (chr. 3, 4, 13, 15. chromosomes 3 and 13 are shown). One chromosome had significantly more upregulated genes in the clone MSC OC.4L.1.4 (chr. 17). Upward (originally red) bars: up-regulation in clone, downward (originally blue) bars: up-regulation in the population. Scale is limited up to −10 or 10 fold.

FIG. 9—Validation of DNA microarray results in MSC OC.4L lineage. Five genes (Xist1, H19, Igf2, Dlx5 and Mest) were selected from the microarray for analysis in real-time PCR. MSC OC.4L lineage cells were subjected to adipogenic induction, and expression of Xist1, H19, Igf2, Dlx5 and Mest, in control and induced cells was examined. The results are presented in the bar graphs shown in FIG. 9, which illustrate the mRNA levels relative to HPRT of the Xist1, H19, Igf2, Dlx5 and Mest genes under the various experimental conditions. All graphs are plotted on logarithmic scale.

FIGS. 10—H4K20me1 global methylation is elevated in OC.4L.1.4 compared to MSC OC. Histone extracts from confluent cells were subjected to Western blotting using a specific H4K20me1 antibody, and densitometry relative to total H4 was calculated. The densitometry results are presented in the left hand bar graph of FIG. 10. A pictogram of the Western Blot experiment is shown in the right hand panel of FIG. 10. Three independent histone extractions were used (one representative blot is shown) for the calculation of methylation amount (1.7 fold increase in MSC OC.4L.1.4, p=0.0267).

FIG. 11—Bar graphs showing H19 knock-down effect on spontaneous adipocyte formation in limiting dilution. Untransfected cells, control transfected cells (i.e., cells transected with non-specific siRNA with similar G:C content) and H19 siRNA transfected MSC OC cells were seeded at limiting dilution in 96-well plates (0.2, 1, 10, 100 and 1000 cells per well: X axis). One month after seeding (with weekly feeding), wells were inspected for adipocyte presence, and wells with one distinguishable adipocyte or more were scored positive. Experiment was done one time in duplicate plates. The percent of adipocyte positive wells from total populated wells is shown.

FIGS. 12A-B—Infection efficiency of MSC OC and MSC OA using lentiviral vector. Plasmid pFUGW was cotransfected with HIV-1 packaging vector Delta8.9 and the VSVG envelope glycoprotein into 293T fibroblasts, and total viral content from supernatant was collected and diluted to the following concentrations: 100%, 50%, 25%, 12.5%, 6.25%, 3.125% and 1.55%. Stock and diluted viral supernatant was applied to MSC OA and OC cultures, and viral infection efficiency was evaluated based on GFP marker using FACS analysis. FIG. 12A shows results of the FACS analysis and FIG. 12B is a graphic representation of infection efficiency (illustrating percent of positive GFP cells (% GFP positive) vs. infection percent (% infection) of MSC OA (dashed line) and MSC OC (solid line) cultures.

FIGS. 13A-D—Acquisition of adipogenic potential occurs on the single cell level. MSC OC was infected with a lentiviral GFP vector as described in FIG. 12. FIG. 13A—pictograms of cells showing adipogenic differentiation of clonal derivations of MSC OCGFP and staining with Oil red O. 11/12 subclones of OCGFP.C fat positive and 5/13 of OCGFP.D subclones fat positive. FIG. 13B (left hand) is a schematic illustration of HindIII restriction map of pFUGW. FIG. 13B (right hand) is a pictogram of Southern blot analysis of MSC OCGFP clones (5 μg of DNA per lane) using DIG labeled probe designed against the LTR regions of the lentiviral insert (88 bp long). pFUGW restricted over-night with HindIII was used as a positive control and two dominant bands (556 bp and 5030 bp) which are complementary to the probe used are evident (unspecific bands due to the large amount of plasmid used or incomplete digestion). MSC OC was used as a negative control. MSC OC GFP was used as a positive internal control. Experiment was performed two separate times using LTR specific probe. FIG. 13C illustrate Bar graphs showing the expression profiles of four adipogenic genes (H19, PPARγ1 (PPARG-1), PPARγ2 (PPARG-2) and EBF), in OCGFP and its clones C/C.4, by real-time PCR. FIG. 13D is a schematic illustration demonstrating LTR probe and the theoretical targets it detects (556 bp internal control and an unknown length target composed of viral and host genomic DNA dependent on gDNA HindIII location downstream of viral insertion).

FIGS. 14A-C MSC OC does not inhibit adipogenic differentiation and spontaneously acquires adipogenic potential in limiting dilution. FIG. 14A is a bar graph showing Oil red O quantification of adipogenic differentiation of MSC OC.4L.1.4 mixed with MSC OC at increasing amounts. FIG. 14B pictograms of Oil red O staining of cell mixtures as in FIG. 14A (from left to right: MSC OC alone, 12.5%, 25%, 50% and 100% MSC OC.4L.1.4). The experiments presented in FIGS. 14A-B were performed two separate times and representative data is shown. FIG. 14C is a bar graph showing the percent of adipocyte positive wells in correlation to type of cell (MSC OC or MSC OCGFP) and number of cells seeded in 96-well plates (0.2, 1, 10, 100, 1000 and 10000). Three separate limiting dilution assays were performed and are summarized in the graph.

FIGS. 15A-B MSC OC and MSC OC.4L.1.4 have significant differences in the histone modification H4K20me1. FIG. 15A—shows pictograms of western blotting using a specific H4K20me1 antibody of Histone extracts from confluent cells (top panel), and bar graphs of calculated densitometry relative to total H4 is MSC OA and MCO.4L.1.4 cells. Three independent histone extractions were used (one representative blot is shown) for the calculation of methylation amount (1.7 fold increase in MSC OC.4L.1.4, p=0.0267, paired t-test). FIG. 15B—Methylation of H4K20me1 at the xist locus as revealed by chip-seq analysis in MSC OC and MSC OC.4L.1.4. As negative control, non-immune serum was used instead of the antibody. Error bars indicate mean±s.e.m.

FIG. 16 Differences in differentiation related and wnt related genes between MSC OC and MSC OC.4L.1.4. Shown in FIG. 16 are graphs of H4K20me1 chip-seq of various genes related to differentiation (left hand panel) or Wnt signaling (right hand panel) of MCS OA or MCS OC.4L.1.4 cell extracts. Each comparison was done using the same scale of peak height. Negative control (non-immune serum) was also performed, but not shown. Wnt related genes wisp1 and ndrg1 are on adjacent chromosomal loci and are shown together.

FIGS. 17A-D Beta-catenin translocates into the nucleus in solitary cells. Shown in FIGS. 17A-C are pictograms of solitary cells (MSC OC, FIG. 17A; MSC OD, FIG. 17B and MSC OM, FIG. 17C), seeded in dense and dilute conditions, fixed and stained with an anti-beta Catenin antibody. left hand column—light microscope view; middle column—fluorescence images of DAPI nuclear staining (originally blue); right hand columns—fluorescence images of cells stained with anti-beta catenin (originally red). Shown in FIG. 17D is a pictogram of a dense culture of MSC OC.

FIG. 18 Adipogenic acquisition in clones derived from MSC OC is lowered by inhibition of wnt signaling and hypoxia. Presented in FIG. 18 are bar graphs showing the adipogenic acquisition (values represent the incidence of adipogenic positive wells in a 96-well plate) of clones derived from MSC OC, by inhibiting wnt signaling (by using sfrp2 at 50 ng/ml) or under hypoxic conditions (3% oxygen), as compared to control, non treated cells.

FIGS. 19A-B MSCs grown from dilute conditions express epithelial and endothelial markers. Shown in FIG. 19 are pictograms of cells, seed in dilute conditions (15,000 cells in a 10 cm plate) that were allowed to re-reach confluency. Cells were then analyzed by immunostaining (FIG. 19A) or by Western blotting (FIG. 19B). FIG. 19A: left hand column—light microscope view; middle column—DAPI nuclear staining (originally blue); right hand columns—fluorescence images of cells stained with an antibody against an epithelial marker (anti-E-cad, originally red). FIG. 19B pictograms of Western blot analysis of protein extracts from cells grown under dense conditions, or after dilution, analyzed with anti vWF (endothelial marker) or anti-E-Cad (epithelial marker). Control protein is GAPDH.

### DETAILED DESCRIPTION OF THE INVENTION

The inventors of the present invention have surprisingly found that isolated clones of mesenchymal stromal cells can de-differentiate and regain differentiative potency by becoming uni-potent cells, bi-potent cells, tri potent cells or otherwise multipotent cells. The dedifferentiated cells so obtained can then differentiate to various cell types/lineages, such as, for example, adipogenic, cohndorogenic, osteogenic, or the like. The inventors of the present invention have further found that, surprisingly, most of bone marrow derived mesenchymal cells do not comply with the definition of MSCs, as they are not necessarily tri-potent. As exemplified herein below, some clonal populations derived from these de-differentiated cells lose subsequently their differentiation potential following long-term culture. As further exemplified hereinbelow cell clones that gain, rather than lose, differentiation potentials have been found. According to some embodiments, and without wishing to be bound by any theory or mechanism, the process, through which cells acquire new differentiation potentials may be triggered by low density culturing and may involve alteration in endogenous gene expression.

According to some embodiments, the present invention is based on the unexpected and surprising finding of methods of inducing de-differentiation of cells, in particular of mesenchymal stromal cells, wherein the methods use mild conditions for the induction and do not involve the use of transfection and/or infection and/or overexpression of exogenous genes in these cells. As further exemplified hereinbelow, the methods of the present invention enable de-differentiation of mesenchymal stromal cells, whereby the cells gain (regain) differentiation potential. In some embodiments, mesenchymal cells that lack differentiation potential altogether (i.e., impotnet cells) gain unipotentiality. In some embodiments, uni-potent cells gain bi-potency or multipotency. In some embodiments, bi-potency cells gain multipoetncy.

According to some embodiments, the present invention is thus directed to novel methods for the induction of dedifferentiation of cells, such as, for example, mesenchymal stromal cells (MSCs), wherein the methods comprise mild conditions and do not involve the use of overexpression of exogenous genes to induce such dedifferentiation of the cells.

### DEFINITIONS

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As used herein, the terms “mesenchymal stromal cell (MSC)” and “mesenchymal stem cell” may interchangeably be used are directed to include any type of mesenchymal stromal cell of any origin. For example, the MSC may be obtained from human, murine, canine, poultry, cattle, farm animals, cats, primates (chimps and other monkeys), birds or any other animal. For example, the MSC may be obtained from various organs or tissues, such as, for example, bone marrow, adipose tissue, spleen tissue, intestine, liver tissue, muscle tissue, brain, skin, ear, bone/cartilage tissues, dental tissue, embryonic tissue, cord blood, placenta, heart, nervous system, spinal cord and the like, or combinations thereof.

As referred to herein, the terms “de-differentiation”, “dedifferentiation” and “reprogramming” may interchangeably be used. The terms are directed to a process of gaining a differentiation potential by a cell. In some embodiments, the terms are directed to the reversion of a cell state/condition to a more generalized or primitive condition. In some exemplary embodiments, the terms are directed to the process whereby a cell becomes uni-potent. In some exemplary embodiments, the terms are directed to the process whereby a cell becomes bi-potent. In some exemplary embodiments, the terms are directed to the process whereby a cell becomes tri-potent. In some exemplary embodiments, the terms are directed to the process whereby a cell becomes multipotent.

As referred to herein, the terms “multipotent”, “multi-potent”, pluripotent and “oligopotent” may interchangeably be used and are directed to cell(s) which has the ability/potency to differentiate to several different lineages, for example, two or more. For example, a multipotent cell may have the potency to differentiate to cells having adipogenic, osteogenic, chondrogenic and/or any other phenotype. In some embodiments, a multipotent cell may be a tripotent cell. In some embodiments, a multipotent cell may be a bi-potent cell.

As referred to herein, the terms “tri-potent” and “tripotent” may interchangeably be used and are directed to a cell which has the ability to differentiate to at least three different lineages. For example, a tri-potent cell may differentiate to cells having adipogenic, osteogenic and chondrogenic phenotypes.

As referred to herein, the term “bi-potent” and “bipotent” may interchangeably be used and are directed to a cell which has the ability to differentiate to at least two different lineages. For example, a bi-potent cell may differentiate to cells having at least two of adipogenic, osteogenic or chondrogenic phenotypes.

As referred to herein, the term “uni-potent” and “unipotent” may interchangeably be used and are directed to a cell which has the ability to differentiate to a designated lineage. For example, a uni-potent cell may differentiate to cells having adipogenic phenotype, osteogenic phenotype or chondrogenic phenotype.

As referred to herein the term “impotent” or “nullipotent” may be used interchangeably and are directed to a cell which has no ability to differentiate.

As referred to herein, the term “pluripotency” refers to the capacity of a cell to differentiate into many tissues and organs excluding a few.

As referred to herein, the term “totipotency” refers to a property of the Zygote that can make all cells of an organism.

As referred to herein, the term “cell density” is directed to the density of cells on a given substrate, plate, well, dish, container, and the like, in which the cells are grown/seeded. The container in which cells are grown/seeded is well known to those skilled in the art and may include, for example, such containers as, but not limited to: tissues culture plates and dishes, tissue culture wells, at various sizes and shapes, which are well known in the art, such as, for example, 384-wells, 96-wells, 48-wells, 24-wells, 12-wells, 6-wells, 100 mm dishes, and the like. In some embodiments, cell density may be measured/expressed in units of number of cells per surface area. For example, cell density may be in the range of, for example, 0-100000 cells per 0.3 cm². For example, the cell density may be about 100 cells per 0.3 cm². In some embodiments, cell density may be expressed as the number of cells per plate, dish, well, and the like. For example, the density may be in the range of, for example, 0-100000 cells per one 96-well size well. For example, the density may, for example, about 60 cells per 96 well or about 15000 cells per 100 mm plate. In some embodiments, cell density may be measured/expressed in units of confluency, i.e. the percentage of coverage of the substrate, plate, well, dish, and the like, in which the cells are grown/seeded. For example, confluency may range from 0% (i.e. no cells) to 100% (i.e. the entire surface area is covered with cells). In some embodiments, density may be expressed/determined as the number of cells per volume. For example, 0-100000 cells per 0.3 cm³. For example, the cell density may be about 1000 cells per 0.3 cm³.

As used herein, the terms “introducing”, “transfection” or “transfecting” and “infection” or “infecting” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, and the like, or combinations thereof. The transfection of the cell may be performed on any type of cell, of any origin.

As referred to herein, the term “exogenous gene” is directed to a gene (or any part thereof) which is introduced from the exterior into a cell. In some embodiments, the exogenous gene is inserted in the form of a polynucleotide (for example, DNA, RNA, and the like). In some embodiments, the exogenous gene is capable of being expressed in the cell. In some embodiments, the exogenous gene is overexpressed within the cell.

As used herein the term “about” in reference to a numerical value stated herein is to be understood as the stated value+/−10%.

According to some embodiments, there are provided methods for the reprogramming of cells by inducing the cells to de-differentiate to other desired cell types, wherein the methods do not include introduction or overexpression of exogenous genes in the cells.

In some embodiments, the cells may be any type of somatic cells, of any origin, such as, for example, human, or animal cell. Examples of somatic cells may include such cells as, but no limited to: epithelial cells, bone marrow cells, fibroblast cells, hepatic cells, hematopoietic cells, intestinal cells, mesenchymal cells, spleen cells, various types of stem cells (such as, for example, blood stem cells, bone stem cells, muscle stem cells, liver stem cells, brain stem cells, and the like. In some embodiments, the cells are not germ line cells.

In some embodiments, the cells are mesenchymal stromal cells. In some embodiments, the mesenchymal stromal cells may be of mouse origin, and their plasticity may be assessed. The mouse MSC may be derived from the mouse bone marrow and may be defined by their tri-lineage differentiation capacity into adipocytes, osteocytes and/or chondrocytes, or any other marker of MSC. In some embodiments, the mesenchymal stromal cells may be of human origin, and their plasticity may be assessed. The human MSC may be derived from the human bone marrow, adipose tissue, or the like, and may be defined by their tri-lineage differentiation capacity into adipocytes, osteocytes and/or chondrocytes, or any other marker of MSC.

According to some embodiments, and as further exemplified hereinbelow, a MSC population of bone marrow mesenchymal cells may be heterogeneous in their differentiation potential. In some instances, some of the MSC are impotent. In some instances, some of the MSCs are unipotent. In some instances, some of the MSCs are bipotent. In some instances, some of the MSCs are tripotent.

According to other embodiments, and as further exemplified herein below, clonal mesenchymal cell populations may be heterogeneous in their differentiation potential. In some embodiments, seeding isolated mesenchymal cells may trigger their reprogramming and allow acquisition of new differentiation potencies (i.e. de-differentiation).

According to some embodiments, there is thus provided a method for inducing dedifferentiation of mesenchymal stromal cell, the method comprising incubating/growing/seeding/plating the cells at low density for a desired period of time and optionally identifying and isolating dedifferentiated cells. In some embodiments, the dedifferentiated cells are single cell derived colonies. In some embodiments, low density is a density of about 10000 cells per 0.3 cm² or less. In some embodiments, low density is a density of about 9000 cells per 0.3 cm² or less. In some embodiments, low density is a density of about 8000 cells per 0.3 cm² or less. In some embodiments, low density is a density of about 7000 cells per 0.3 cm² or less. In some embodiments, low density is a density of about 6000 cells per 0.3 cm² or less. In some embodiments, low density is a density of about 5000 cells per 0.3 cm² or less. In some embodiments, low density is a density of about 4000 cells per 0.3 cm² or less. In some embodiments, low density is a density of about 3000 cells per 0.3 cm² or less. In some embodiments, low density is a density of about 2000 cells per 0.3 cm² or less. In some embodiments, low density is a density of about 1000 cells per 0.3 cm² or less. In some embodiments, low density is a density of about 900 cells per 0.3 cm² or less. In some embodiments, low density is a density of about 800 cells per 0.3 cm² or less. In some embodiments, low density is a density of about 700 cells per 0.3 cm² or less. In some embodiments, low density is a density of about 600 cells per 0.3 cm² or less. In some embodiments, low density is a density of about 500 cells per 0.3 cm² or less. In some embodiments, low density is a density of about 450 cells per 0.3 cm² or less. In some embodiments, low density is a density of about 400 cells per 0.3 cm² or less. In some embodiments, low density is a density of about 350 cells per 0.3 cm² or less. In some embodiments, low density is a density of about 300 cells per 0.3 cm² or less. In some embodiments, low density is a density of about 250 cells per 0.3 cm² or less. In some embodiments, low density is a density of about 200 cells per 0.3 cm² or less. In some embodiments, low density is a density of about 150 cells per 0.3 cm² or less. In some embodiments, low density is a density of about 100 cells per 0.3 cm² or less. In some embodiments, low density is a density of about 50 cells per 0.3 cm² or less. In some embodiments, low density is a density of about 25 cells per 0.3 cm² or less. In some embodiments, low density is a density of about 10 cells per 0.3 cm² or less. In some embodiments, low density is a density of about 5 cells per 0.3 cm² or less. In some embodiments, low density is a density of about 2 cells per 0.3 cm² or less. In some embodiments, low density is a density of about 1 cell per 0.3 cm² or less. In some embodiments, low density is a density of about 0.5 cell per 0.3 cm² or less. In some embodiments, low density is a density of about 0.25 cell per 0.3 cm² or less. In some exemplary embodiments, low density is in the range of about 0.1-5000 cells per 0.3 cm². In some embodiments, low density is a density in the range of about 0.2-2000 cells per 0.3 cm². Each possibility is a separate embodiment.

According to some embodiments, the period of time in which the cells are grown in low density may range from about 4 hours to about 8 weeks. In some embodiments, the period of time may range from about 4 hours to about 12 hours. In some embodiments, the period of time may range from about 4 hours to about 24 hours. In some embodiments, the period of time may range from about 4 hours to about 48 hours. In some embodiments, the period of time may range from about 4 hours to about 72 hours. In some embodiments, the period of time may range from about 4 hours to about 96 hours. In some embodiments, the period of time may range from about 4 hours to about 120 hours. In some embodiments, the period of time may range from about 4 hours to about a week. In some embodiments, the period of time may range from about 4 hours to about two weeks. In some embodiments, the period of time may range from about 4 hours to about three weeks. In some embodiments, the period of time may range from about 4 hours to about four weeks. In some embodiments, the period of time may range from about 4 hours to about five weeks. In some embodiments, the period of time may range from about 4 hours to about six weeks. For example, the period of time may be in the range of about 96 hours.

In some embodiments, identifying cells that are dedifferentiated may be performed by various methods, such as, for example, but not limited to: visual identification of acquired phenotype (such as, morphology), identification of expression of specific markers unique to the dedifferentiated state, identification of molecular markers, identification of modulation of gene expression, and the like. For example, appearance of adipogenic cells within a population, which a prioi lacked this potential, is observable at a single cell level, hence acquiring dedifferentiation potential into this type of cells can be followed with high accuracy and sensitivity. For example, cells may become epithelial-like or endothelial-like cells. For example, reprogrammed cells may show specific changes in gene expression correlated with epigenetic modulations (such as changes in the expression of imprinting genes (such as, Xist, Asb4, Dlx5, Mest, H19, Igf2, and the like); changes in their posttranslational modification (for example, elevated H4K20me1 total histone methylation in reprogrammed cells); and the like. For example, reprogrammed cells may show specific changes in gene expression profiles, such as changes in differentiation and pluripotency related genes (such as, but not limited to: nestin, Cebpδ, lp1, angpt1, kit1, gas6, svep1, sned1, cebp alpha, hgf, alp1, sox9, pparγ1, and the like); wnt signaling pathway related genes (such as, but not limited to: ndrg1, pappa2, wisp1, lrp8, wnt 10b, wnt5b, lrp5, rspo2, sfrp2, sfrp1, fzd3, wnt5a, wisp2, lrp4, snai2, lrp11, lrpap1, fzd1, fzd5, lef1, and the like); genes related to insulin pathways (such as, but not limited to:Cxcl5 and Steap4); imprinting genes (such as, but not limited to: Dio3); genes involved in bone and cartilage formation (such as, but not limited to: BMP4); small nucleolar RNAs (such as, but not limited to: snora44, snord118, snord116, snord115, and the like); and the like.

In some embodiments, the methods of inducing de-differentiation of mesenchymal stem cells comprise incubating/growing/seeding the cells at low density (for example, at densities detailed above herein), and, optionally or alternatively may further include varying one or more physical and/or chemical environmental conditions in which the cells are grown. For example, the physical and/or chemical environmental conditions that may be modified, may be selected from, but not limited to: growth medium (for example, high/low salt concentration, ionic strength, glucose concentration, percentage and type of serum used, temperature, CO₂ concentration, O₂ concentration, pressure, humidity, pH, type of substrate(s) on which the cells are grown, type and size of plate/well, irradiation with various irradiation types (for example, UV, X-ray) at various intensities and dosages; cell passage (i.e., the number of generations the cells have been divided), use of various chemical agents (such as, for example, 5-azacytidine), use of various compounds, such as, for example, growth factors (PDGF, GH, IGF, FGF, HGF, EGF, and the like), cytokines, TLR ligands, Wnt inhibitors/activators (such as, for example, sfrp's/wnt's), Notch inhibitors/activators, GSK-3 inhibitors/activators (insulin/lithium), and the like, or combinations thereof.

According to some embodiments, changes/modifications in culture conditions, such as, for example, modifications in the physical parameters of the culture or chemical changes in the growth medium may enhance the frequency and extent of such reprogramming events. According to some embodiments, a combination of such modifications may be used in combination with or alternative to incubating/growing/seeding the cells at low density, to yield specific types of de-differentiated potent cells that are capable of differentiating into desired mature cell types. According to some embodiments, the changes/modifications in culture conditions may be selected from, but not limited to: modification of cell surfaces: elasticity, rigidity, coating culture surfaces with extracellular matrix (ECM) components, use of tri-D matrixes, addition of cytokines and hormones, treating the cells with toll-like receptor ligands (TLRs) or with their inhibitors, co-culturing the cells with tissue slices and injured tissue fragments or their conditioned media, any other modification (such as detailed aboveherein), or the like, or any combination thereof. Additionally, any other modifications in the physical parameters of the culture or chemical changes in the growth medium that are known in the art may be used. In some embodiments, the modifications in the physical parameters of the culture or chemical changes in the growth medium may induce cell stress. Exemplary modifications in the physical parameters of the culture or chemical changes in the growth medium that may affect mesenchymal cells can be found, for example, in Ref 12 (Pevsner-Fischer and Zipori, hereinbelow), or in U.S. Pat. No. 5,942,225, the content of both is incorporated by reference herein in their entirety.

According to some exemplary embodiments, and as further demonstrated herein below, using the methods of the invention, bi-potent mesenchymal cells that were able to differentiate only into osteocytes and chondrocytes, may gain de-differentiation potential and may now become multipotent as they acquire epithelial and endothelial morphologies as well as adipogenesis capability. In other exemplary embodiments, cells that lack chodrogenic potential, regain it by using the methods of the present invention.

According to some embodiments, the reprogramming event of the MSC may occur at a single cell level as exemplified herein by using lentiviral marking for lineage tracing.

According to some embodiments and without wishing to be bound to any theory or mechanism, the de-differentiated cells obtained by the methods of the present invention may differ from the original respective cells in one or more of the following characteristics: phenotypically, they may appear and behave differently (i.e., differentiation capacity changed); transcriptionally (i.e. their gene expression profiles are changed); and/or epigenetically (for example, by undergoing modulation of their histone modifications).

According to some embodiments, and without wishing to be bound to any theory or mechanism, the involvement of small nucleolar RNAs (snoRNAs) is implicated as a molecular mechanism, which may drive cell reprogramming at low density culturing.

According to some embodiments, and without wishing to be bound to any theory or mechanism, the involvement of changes in the wnt signaling pathway is implicated as a molecular mechanism, which may drive cell reprogramming at low density culturing.

In some embodiments, the methods for inducing de-differentiation of mesenchymal stem cells exclude the introduction or expression of exogenous gene(s) in the mesenchymal stromal cells or any other artificial genetic manipulation/modification of the cells.

In some embodiments, dedifferentiated cells, acquired by the methods of the present invention may further be used to provide a desired cell type to be used in various applications, such as, for example, tissue regeneration.

In some embodiments, dedifferentiated cells, acquired by the methods of the present invention may further be introduced to a host for various purposes, such as, for example, for the purpose of mesenchymal tissue regeneration, tissue repair, and the like. In some embodiments, the dedifferentiated cells, acquired by the methods of the present invention may further be introduced to a host from which they originated.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Materials and Methods

### BM Cell Extraction and MSC Production

BM cells were obtained from 8-10 week old C57BL/6 mice: the tips of the bones (femur and tibia) were gently cut and the bone was flushed with phosphate buffered saline (PBS) containing 1% FCS. The cells were dissociated to single cell suspension and centrifuged at 1200 g for 5 min. The pelleted cells were re-suspended to single cell suspension and seeded in two wells in a 6-well plate (Falcon) containing MSC medium (Stem cell technologies). Half of the medium was replaced every 3 days to remove the non-adherent cells. Once the adherent cells had reached confluence, the cells were trypsinized using Trypsin B solution (0.05% EDTA, 0.25% trypsin), centrifuged for 5 min at 1200 g, re-suspended in their medium and split 1:2.

### Flow Cytometry Analysis

For flow cytometry analysis the following antibodies were used: anti-CD 11b-PE, anti-CD45.2-PE, and anti-SCA-1-PE were purchased from Southern Biotech. Rat IgG2a isotype control-RPE was purchased from eBioscience. MSCs were harvested, washed once in cold PBS containing 0.5% bovine serum albumin (BSA) and 0.03% sodium azid, and incubated with specific antibodies for 1 hour. Next, cells were washed and subjected to flow cytometric analysis using a FACScan flow cytometer (Becton Dickinson Immunocytometry System, San Jose Calif.). Cells were gated according to their high fluorescence intensity using Cell Quest analysis software (Becton Dickinson).

### Evaluation of Stromal Cell Differentiation

Adipogenesis:

Cells were seeded at a concentration of about 30,000 cells per well in a 24 well plate (falcon) in growth medium containing DMEM supplemented with 10% FCS. The next day growth medium was replaced with differentiation medium containing DMEM supplemented with 10% fetal bovine serum (FBS, HyClone), insulin (10 μg/ml, Sigma), isobutylmethylxanthine (IBMX, 0.5 mM, Sigma) and dexamethasone (1×10⁻⁵M, Sigma). The cells were grown for one to three weeks, with medium replacement twice a week. Adipogenesis was detected by Oil red O staining Fresh Oil red O working solution was prepared in each staining procedure by mixing 60% oil red O stock solution (0.5% oil red O, Sigma, in 100% isopropanol) with 40% water. Cells were washed twice with PBS and fixated with 4% PFA for 20 min at room temperature (RT), washed again and stained with Oil red O working solution for 10 min in RT. For Oil red O quantification, 4% IGEPAL CA 630 (sigma) in isopropanol was added to each well for 15-30 min. Light absorbance by the extracted dye was measured in 492 nm.

Osteogenesis—

Cells were seeded at a concentration of about 30,000 cells per well in a 24 well plate (falcon) in growth medium containing DMEM supplemented with 10% FCS. The next day growth medium was replaced with differentiation medium containing DMEM supplemented with 10% FBS, L-Ascorbic acid-2 phosphate (50 μg/ml, Sigma), glycerol 2-phosphate di-sodium salt (10 mM, Sigma), and dexamethasone (1×10⁻⁷M, Sigma. The cells were grown for one to three weeks, with medium replacement twice a week. Osteogenic differentiation was detected by alizarin red staining Cells were washed twice with PBS and fixated with 4% paraformaldehyde (PFA) for 15 min, washed again, and stained with 2% alizarin red solution for 15 min in RT. For alizarin red quantification, 0.5N HCl, 5% sodium dodecyl sulfate (SDS) was added to each well for 5-10 min. Light absorbance by the extracted dye was measured in 405 nm.

Chondrogenesis—

For chondrogenesis, cells were grown in micro-mass culture supplied with chondrogenesis induction medium. 0.2×10⁶ cells were centrifuged 5 min at 1200 g in 15 ml conical polyproylane tubes. After centrifugation, the supernatant was gently removed and 1 ml of chondrogenesis medium containing: L-ascorbic acid-2 phosphate (0.1 mM), human TGF-β1 (10 ng/ml, Peprotech/Cytolab), dexamethasone (1×10⁻⁷M) was added. The tubes were incubated with the cap slightly open for 3 weeks, with medium replacement twice a week. The pellets were washed and fixed with 4% PFA for 1.5 hour. The samples were embedded in 3% agarose (sigma, A9045) followed by paraffin embedding. Chondrogenic differentiation was detected by alcian blue staining: Paraffin-embedded sections were deparaffinized in xylene and rehydrated in graded alcohol. The sections were washed twice with distilled water, stained with alcian blue solution and counterstained with nuclear fast red solution. The sections were washed again in running tap water and then dehydrated by incubations in increasing alcohol concentrations: 70, 95, 100% followed by incubation with xylene, and finally mounted with entellan.

Isolation of Clones from Primary MSCs

MSCs derived as above were subjected to clonal isolation using limiting dilution. MSCs at passages 5-7 were seeded in 96-well plates (falcon) at a concentration of 0.25 cells/well and grown in MSC medium. Colonies formed were observed under a light microscope (Olympus CK-2) and only those which originated from a single cell were passed on to 24-well plates (falcon). Once reaching confluence, cells were passed to 60 mm plates (falcon), and subsequently were frozen in aliquots. For differentiation evaluation, cells were treated as described above with the following changes: 10,000 cells per well were seeded in 96-well plates, which were coated with fibronectin (20 μg/ml) overnight, and grown in MSC medium. After 3 days, medium was changed to differentiation medium.

### Western Blot

Proteins were extracted on ice for 10 minutes using RIPA buffer (50 mM Tris HCl pH 8, 150 mM NaCl, 1% NP-40, 0.5% sodium Deoxycholate, 0.1% SDS) supplemented with 1:100 protease inhibitor. Proteins were loaded and fractionated onto 10% SDS-PAGE. For histone analysis, nuclear fractions of equal amounts of confluent cells were extracted (−10⁶ cells) according to the Abcam acid extract protocol. Immunoblot analysis was performed with anti-GFP (Clontech 1:200), anti-H4k20me1 (Abcam ab9051 1:1000), anti-H4 total (Cell signaling #2935 1:1000), anti-vWF (Millipore AB7356 1:1000), anti GAPDH (sigma 1:10,000) or anti-E-cadherin (cell signaling #3195 1:1000). Specific binding was detected with horseradish peroxidase (HRP)-coupled antibody and enhanced chemiluminescence (ECL) reagent.

### Chromatin Immunoprecipitation Sequencing (Chip-Seq)

Formaldehyde (BIO LAB Ltd.) was directly added to confluent cultured MSCs (1-6×10⁶) plates for a final concentration of 1% for 10 min at RT. Glycine (Sigma) was added to a final concentration of 0.125M for 5 min at RT. Two washes with PBS and collect to tubes with PBS, protease inhibitor cocktail (1:100, PI, Sigma), Pepstatin (1:1000, Pep, Sigma), and centrifuged at 700 g, 4 C for 5 min. Pellet was suspended in 2.5 mL buffer B (20 mM Hepes pH 7.5 (Sigma), 0.25% Triton-X100 (Sigma), 10 mM EDTA (J.T.Barker), 0.5 mM EGTA (J.T.Barker), PI (1:100), Pep (1:1000)), rotated for 10 min on ice and centrifuged at 500 g, 4 C for 5 min. This step was repeated again using buffer C (50 mM Hepes pH 7.5, 150 mM NaCl, 1 mM EDTA, 0.5 mM EGTA, PI (1:100), Pep (1:1000)). Finally, the pellet was suspended in 300 ul Lysis buffer (1% SDS (inno-TRAin), 10 mM EDTA, 50 mM tris-HCl pH 8.1, PI (1:100), Pep (1:1000)). The cell lysate was subjected to sonication for 45 min at maximum intensity using Bioraptor™ (Wolf Laboratories Ltd, USA). The supernatant was pre-cleared as following: 300 ul of sonicated DNA were mixed with 1200 ul of cold dilution buffer (20 mM tris-HCl pH 8.1, 2 mM EDTA, 150 mM NaCl, 1% Triton-X100) and 40 ul washed Agarose-Protein A beads (washed three times with TE buffer (10 mM tris-HCl pH 8.1, 1 mM EDTA) (Santa Cruz Biotechnology)), and incubated for 2 h at 4° C. with rotation. The supernatant was collected after spin at 1500 rpm at 4 C. BSA (Sigma) was added to the supernatant to a final concentration of 0.1 mg/ml. For half of the samples IgG anti rabbit non-immune serum (NIS, 4 ug per 1×105 cell, extracted and purified at the lab of Prof Yoram Groner, Weitzman Institute, Israel) was added, and to the other half anti-H4k20me1 (4 ug per 1×105 cell, Abcam) was added. The mix was incubated over-night at 4° C. with rotation. 50 ul of tRNA (10 mg/ml, Sigma) was added to each sample with 40 ul washed beads and rotated for 2 h at 4° C. The supernatant was discarded after spin at 1500 rpm for 2 min. The beads were washed sequentially with 10 ml TSE-150 buffer (20 mM tris-HCl pH 8.1, 2 mM EDTA, 1% Triton X-100, 0.1% SDS, 150 mM NaCl, PI (1:100)), 10 ml TSE-500 buffer (20 mM tris-HCl pH 8.1, 2 mM EDTA, 1% Triton X-100, 0.1% SDS, 500 mM NaCl, PI (1:100)), 10 ml buffer III (10 mM tris-HCl pH 8.1, 250 mM LiCl (Sigma), 1% NP-40 (Sigma), 1% deoxycholate (Sigma), 1 mM EDTA) and twice with TE buffer. Between each wash the beads were rotated at RT for 5 min, centrifuged and discard the supernatant. For the two samples (NIS and antibody), 300 ul Elution buffer (200 ul SDS 20%, 400 ul 1M NaHCO3 (J.T.Barker), 3.4 ml sterile water) was added to each and mixed gently, and incubating at RT. The agarose beads were pelleted (1500 rpm for 2 min) and the eluates were collected, these two steps repeated twice. Added to each sample: 16 μl NaCl 5M, 8 ul EDTA 0.5M, 16 ul tris-Hcl 1M pH 6.5. The samples were incubated over-night at 65 C. 1 ul proteinase K (20 mg/ml, Sigma) was added and incubated for additional 2 h at 55 C. The DNA was eluted using the MinElute® PCR Purification Kit (Qiagen). Pico-drop machine (Invitrogen) was used for DNA concentration determination using Qubit® dsDNA HS Assay Kit (Invitrogen). For sequencing and further bioinformatic analysis the samples were sent to the bioinformatics department, DNA analysis unit at the WIS.

### Real Time PCR and DNA Array

Total RNA was extracted using Tri-Reagent (MRC, Cincinnati, Ohio), according to the manufacturer standard protocol. cDNA was prepared using M-MLV Reverse Transcriptase enzyme (Promega) using manufacturer protocol. All samples were treated with TURBO DNA-Free™ Kit (Ambion). Real time PCR was done usingPlatinum® SYBR® Green qPCR SuperMix-UDG with ROX based assay (invitrogen) and processed by ABI 7300 machine (Applied Biosystems, Lincoln Center, Calif.). Primers were designed using Primer express 3.0 program and the UCSC website. Primers are listed in Table 1, below.

TABLE 1
Primers used for Real time PCR

| Gene | Forward primer | Reverse primer |
|---|---|---|
| HPRT | GCAGTACAGCCCCAAAATGG | GGTCCTTTTCACCAGCAAGCT |
| | (SEQ ID NO. 1) | (SEQ ID NO. 2) |
| pparγ1 | AACAAGACTACCCTTTACTGAAATTACCA | AATGGCATCTCTGTGTCAACCA |
| | (SEQ ID NO. 3) | (SEQ ID NO. 4) |
| pparγ2 | GCATGGTGCCTTCGCTGA | TGGCATCTCTGTGTCAACCATG |
| | (SEQ ID NO. 5) | (SEQ ID NO. 6) |
| znf423 | CCAGTGCCCAGGAAGAAGAC | CGAACGTCATCTGGCACTTG |
| | (SEQ ID NO. 7) | (SEQ ID NO. 8) |
| Ebf1 | CAGCAATGGGATACGGACAGA | GAGTCGATGAGGCGCACATAG |
| | (SEQ ID NO. 9) | (SEQ ID NO. 10) |
| H19 | GCTAGGGTTGGAGAGGAATGG | AAAAGTAACCGGGATGAATGTCTG |
| | (SEQ ID NO. 11) | (SEQ ID NO. 12) |
| Xist | GAGAGAGCCCAAAGGGACAAA | TGGCAGTCCTTGAGTCTCACATAG |
| | (SEQ ID NO. 13) | (SEQ ID NO. 14) |
| IGF-2 | CCGTACTTCCGGACGACTTC | GACTGTCTCCAGGTGTCATATTGG |
| | (SEQ ID NO. 15) | (SEQ ID NO. 16) |
| Mest | TCGTGTTCTCTCGAGGTCTCACT | TGGGTCGAGTATACGGTCCAA |
| | (SEQ ID NO. 17) | (SEQ ID NO. 18) |
| D1x5 | GACTGACGCAAACACAGGTGAA | GATCTTGGATCTTTTGTTCTGAAACC |
| | (SEQ ID NO. 19) | (SEQ ID NO. 20) |
| p53 | CACGTACTCTCCTCCCCTCAAT | AACTGCACAGGGCACGTCTT |
| | (SEQ ID NO. 21) | (SEQ ID NO. 22) |

DNA Mouse Gene ST microarray (Affymetrix®) analysis was performed using 100 ng of total RNA.

### DIG Labeling and Southern Blot

DIG labeling kit (Roche Applied Science) was used for preparing detection probes. Genomic DNA was extracted from target cells using GenElute™ Mammalian Genomic DNA Miniprep kit (Sigma) and digested overnight at 4° C. using HindIII, BamHI or MfeI (Fermentas). Digested DNA and probes were hybridized as instructed by Roche's DIG manual. 2-10 μg of DNA was loaded on agarose gel, and detection was done using DIG detection kit (Roche).

### Transfection and Lentiviral Infection

Sub-confluent 293T cells were transfected with FUGW, VSVG and Δ8.9 plasmids using CaCl₂ and HBSS. Supernatant containing viral particles was collected 48 hours after transfection, filtered and applied to target cells for infection. Infection efficiency was determined using a fluorescent microscope and FACS for the detection of GFP positive cells.

### Fluorescent Staining

For OCT4 staining cells were layered on glass slides using a cytospoin machine (Shandon elliott). The cells were fixed with 4% PFA for 10 min, blocked and incubated with a rabbit anti OCT4 antibody (Abcam, ab19857) in blocking solution overnight at 4° C. The next day, the slides were incubated with secondary antibody Alexa Fluor 488 dye 1:000 in blocking solution (Jackson). For beta-catenin staining, cells were grown on glass coverslips coated with fibronectin (Sigma). For E-cadherin staining cells were grown on plastic culture dishes (Falcon). The cells were fixed and blocked as described above and incubated with a rabbit anti beta-catenin antibody (Sigma, c2206), or a rabbit E-cadherin antibody (Cell signaling, #3195) in blocking solution overnight at 4° C. The day after, the slides were incubated with secondary antibody Cy3 (Jackson) at 1:000 in blocking solution. 10 μl of DAPI II (VYSIS) was used for mounting and nuclear staining Photographs were taken using a Zeiss Axio Imager.Z1 microscope (Carl Zeiss MicroImaging GmbH, Germany).

### Example 1

### Bone Marrow Derived Stromal Cells have Variable Differentiation Potentials

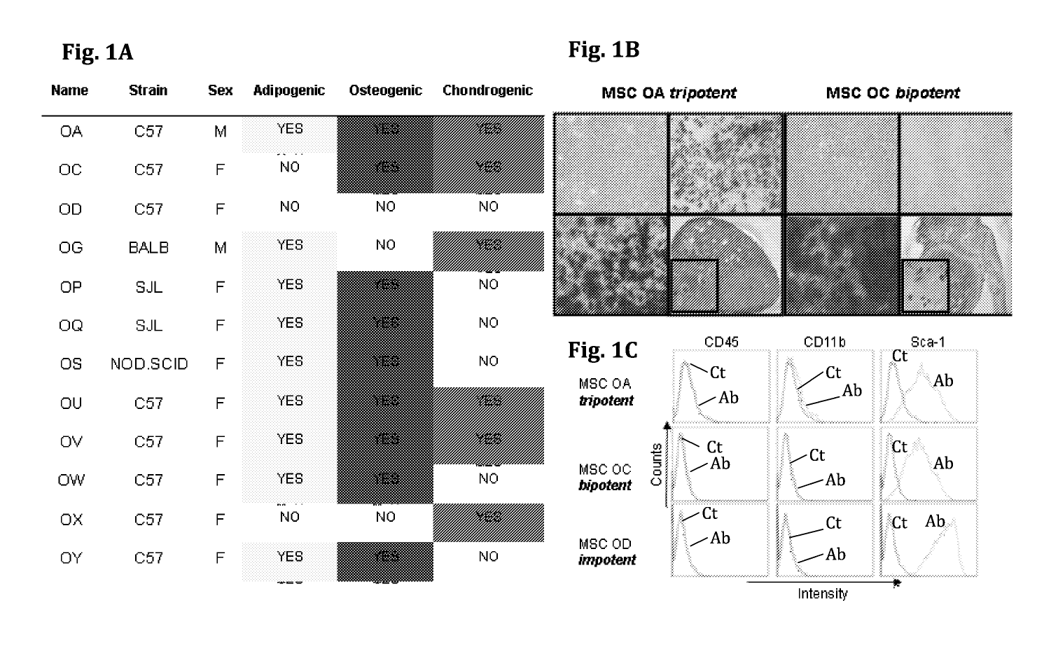

The classical definition of mesenchymal stromal cells (MSCs) states that these cells adhere to plastic, show a fibroblastic morphology in culture, and harbor a tri-potent differentiation potential, being able to acquire adipogenic, osteogenic and chondrogenic phenotypes. To test whether all mesenchymal cells derived from mouse bone marrow follow these rules, the following experiments were performed: 12 independent derivations were examined for their differentiation into adipocytes, osteocytes and chondrocytes in induction media. As shown in FIG. 1.A, of the 12 different mesenchymal cell preparations that were derived, only three showed a tri-potent differentiation potential, whereas all other derivations exhibited variable potencies. These findings show that there is vast heterogeneity in cultured mesenchymal cells, even though they were all derived under exactly the same conditions. As shown in FIG. 1B, which demonstrates pictographs of staining with Oil red O (adipocytes), alizarin red (osteocytes) and Alcian blue (chondrocytes) markers of mesenchymal cells derivation, the mesenchymal derivation, termed herein MSC OC, has an osteogenic-chondrogenic differentiation potential (i.e., is bi-potential), and does not have any adipogenic potential (right hand panel), as compared to MSC OA, mesenchymal derivation, which is a tri-potent derivation (left hand panel). The MSC OC derivation does not differentiate to adipogenic phenotype, even when induced with TGFβ2 and/or BMP4, molecules which are implicated in the induction of adipogenic differentiation (beside the regular cocktail used—Recombinant TGF-β2, BMP4 proteins were added separately or together to serum-free culture medium or differentiation cocktails were added at a concentration of 10 ng ml⁻¹ for all experiments). The MSC OC did not show even the slightest adipogenic potential under these different conditions and after continued passaging in vitro, and was assayed at twelve different occasions. As shown in FIG. 1C, surface marker analysis by FACS verified that the MSC derivations obtained are not contaminated with hematopoietic cells and regardless of their differentiation potential, they express the MSC marker, Sca-1, and not CD45 (hematopoietic cell marker), or CD11b (macrophage marker).

### Example 2

### Multipotent Differentiation of MSCs after Low Density Seeding

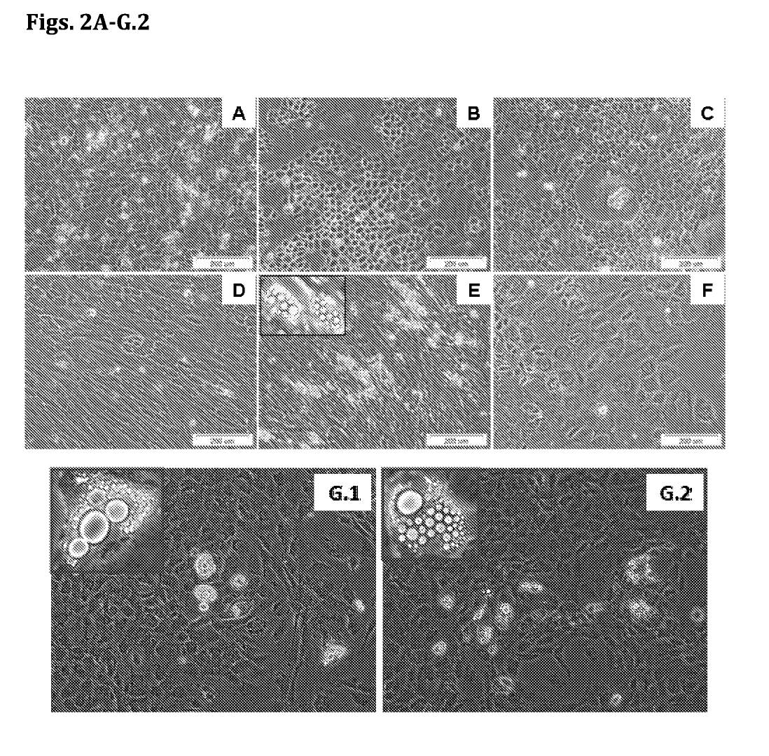

Next, the heterogeneity of mesenchymal cells was tested on specific clones. The results, shown in the pictograms of FIG. 2, demonstrate that the heterogeneity of mesenchymal cells is not restricted to whole population analyses. As shown in FIG. 2B, low density seeding of MSC OC (i.e., MSC OC cells after two rounds of low density seeding (first 15,000 cells in 100 mm dish reached confluence, collected, reseeded at 2,500 cells in 100 mm dish and allowed to reach confluence)) resulted in clonal expansion and the appearance of various cell morphologies characteristic of epithelial-like cells, and of endothelial-like cells, shown in FIG. 2F. Surprisingly, some cells in the culture became fat laden implying acquisition of adipogenic potential (as shown in FIG. 2E), which is absent in the original cell population (FIG. 2A, MSC OC seeded at normal density (1:4 passage), was allowed to reach confluence). The appearance of giant cells (FIG. 2C) and spindle shaped fibroblasts (FIG. 2D) were also evident in the culture. Other low-density cultures ((2,500 cells seeded in 100 mm dish) gave similar results, for example, MSC OD which is lacking any differentiation potency, acquired an adipogenic phenotype, as shown in FIG. 2.G.1/2).

### Example 3

### MSC Clones Lose and Gain Differentiation Potentials

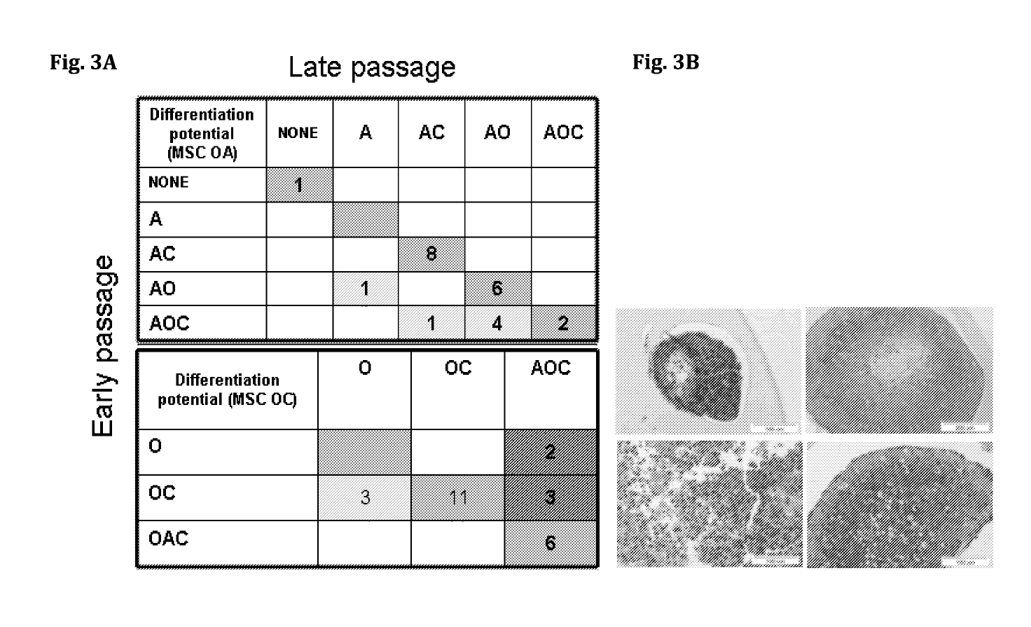

Since only in the low-density culture newly derived cell phenotypes were revealed, may imply that the passaging procedure itself induces/causes the phenomenon. To this aim, clonal analyses of MSCs were performed. MSC OA and MSC OC derivations were seeded at a concentration of 0.2 cells/well in 96-well plates, and single cell derived clones (verified by microscopic view) were grown. Such clones were grown in culture to reach low (three passages after single cell seeding) and high passaged (ten additional passages in culture) clones, and their differentiation potentials were examined. As shown in FIG. 3A, top table, MSC OA clones showed variable differentiation potentials, and only 7 out of 23 clones were found to be tri-potent at the early passage. Five clones have lost their osteogenic or chondrogenic potentials at the late passage and became bi-potent. As shown in FIG. 3A, lower table, cellular cloning of MSC OC, however, gave rise to 6 tripotent clones with a newly acquired adipogenic potential from a total of 25 clones. This clonal adipogenic potential was increased in the late passage clones, as five more clones have gained this property. As further shown in the pictograms of FIG. 3B, two of the MSC-OC clones have also gained a chondrogenic differentiation potential during passaging (as the two clones were negative for chondrogenic differentiation at an early passage, and were found positive by alcian blue staining at the later passage). The clonal acquisition of differentiation potentials further demonstrate that the low density seeding is indeed responsible for such cellular plasticity.

### Example 4

### Lineage Tree of an MSC OC (MSC OC.4-MSC-OC Clone 4) and Differentiation Potential Thereof

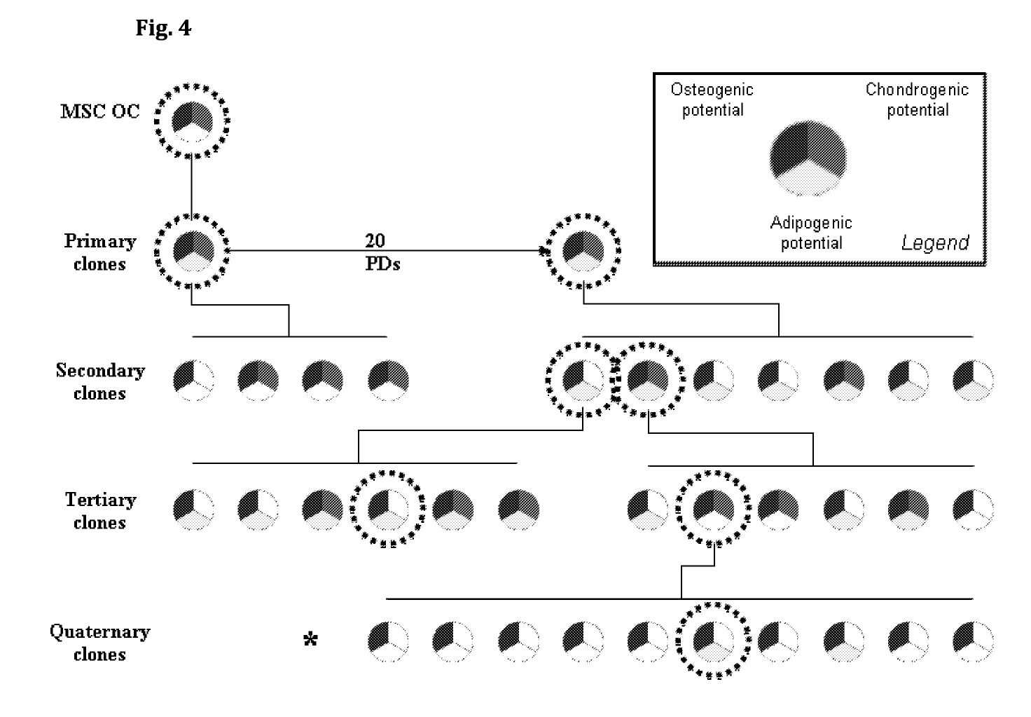

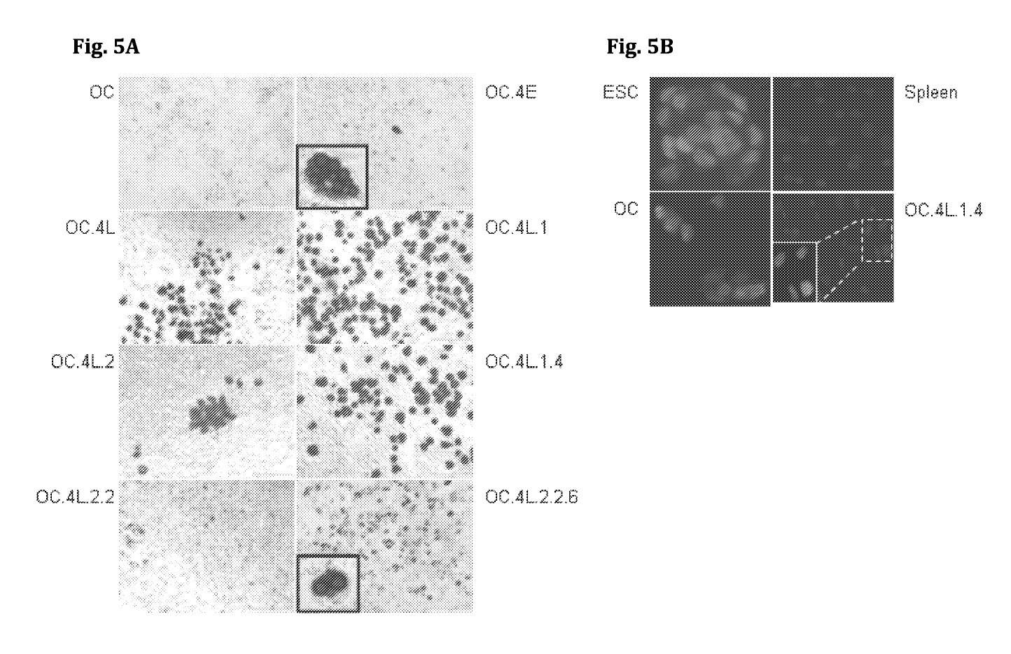

To further delineate the process of acquisition of differentiation potencies following cloning, adipogenesis, which can be followed in real time at the single cell level, was studied. A serial clonal assay was performed, in which the cellular cloning procedure (seeding of 0.2 cells/well) was repeated, until reaching quaternary clones (As illustrated in FIG. 4). The process of cellular cloning was repeated with selected sub-clones until reaching quaternary clones. All clones were subjected to tri-lineage differentiation assays (“red” (black in FIG. 4)—osteogenesis, “yellow” (light gray in FIG. 4)—adipogenesis, “blue” (gray in FIG. 4)—chondrogenesis). Circled clones are selected clones which show the loss and gain of adipogenic potential. Horizontal line represents passaging in culture (PD—population doublings).). As can be observed in the circled clones shown in FIG. 4, the adipogenic potential was not stable, and repeated cloning of the cells led to its acquisition or disappearance. The adipogenic differentiation of the clones circled in FIG. 4 was further tested. As shown in the pictograms of FIG. 5A, (which present MSC OC cell population and its descendent clones, assayed for adipogenic differentiation and stained with Oil red O), MSC OC has no adipogenic potential, whereas its primary clone, OC.4 acquired such a potential at the early passage (OC.4E), which increased at the late passage (OC.4L). All secondary clones derived from OC.4L showed an adipogenic potential (FIG. 4), two of them depicted in FIG. 5A, OC.4L.1 with a high adipogenic potential, and OC.4L.2 with a low adipogenic potential. Tertiary clone OC.4L.1.4 showed an even higher adipogenic potential, however, the tertiary clone OC.4L.2.2 lost the ability to differentiate into adipocytes. The Quaternary clone OC.4L.2.2.6 has re-acquired the ability to differentiate into adipocytes. The acquisition of new cellular traits can be addressed to a reprogramming event, and indeed, as shown in FIG. 5B, which show pictographs of MSC OC and MSC OC.4L.1.4 that were seeded at low density (100,000 cells in 100 mm plate) and stained for OCT4 one day after seeding, MSC OC and MSC OC.4L.1.4 show positive staining for OCT4 (a well known pluripotency marker usually associated to embryonic stem cells), when cultured at low density.

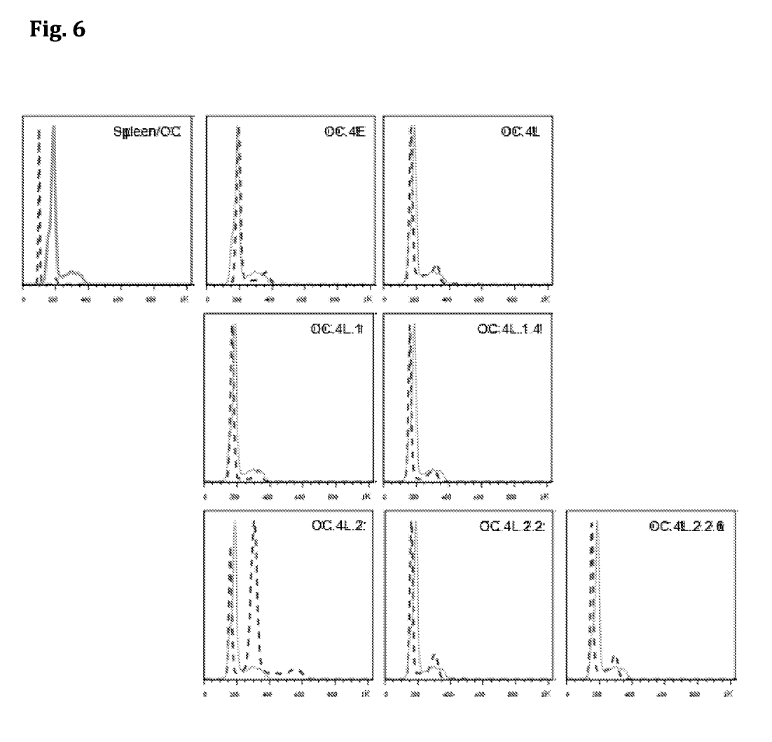

It is known that cells in culture have a tendency towards wide genome re-arrangements, such as total chromosomal number alterations. Such wide genomic changes might play a part in the plasticity exhibited by the MSC cells. For this reason, the ploidy of the different clones in the MSC OC lineage was examined and compared to control diploid primary spleen cells. As shown by the FACS analyses presented in FIG. 6, MSC OC appear to be near tetraploid, and none of its clones (with the exception of MSC OC.4L.2) has changed its ploidy in comparison, indicating there is no clear correlation between such large scale genomic alterations and the plasticity observed in the cells.

### Example 5

### Acquisition of Adipogenic Potential is Accompanied by Changes in Adipogenic Gene Expression and Epigenetic Modulation

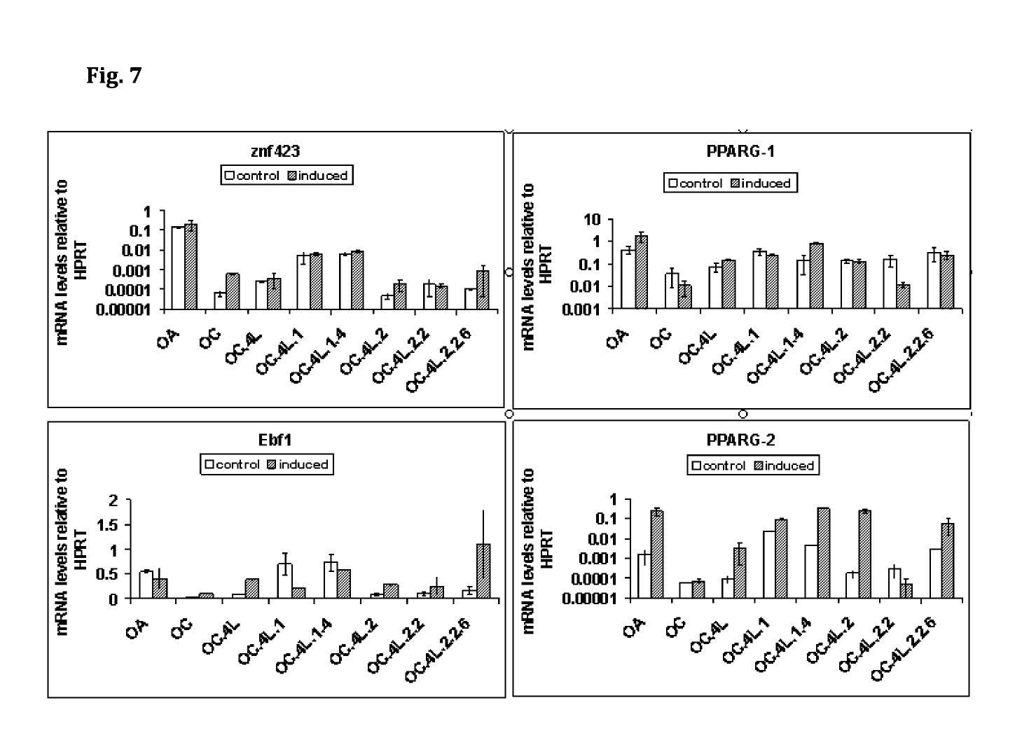

The adipogenesis process has a well-defined underlying molecular theme, which involves many proteins such as, for example, the C/EBP family and PPARγ 1/2. Reference is made to FIG. 7, which shows bar graphs of expression of 4 marker genes as determined by real-time PCR analysis of MSC OC and its derivative clones, before and after adipogenic induction in culture (control/induced). As shown in FIG. 7, compared to the adipogenic MSC OA, MSC OC shows a much lower basal expression profile of PPARγ 1 and 2 and is non-reactive to adipogenic induction. In contrast, the adipogenic clones OC.4L.1 and OC.4L.1.4 showed an elevated basal PPARγ 1/2 expression, and after adipogenic induction the expression was even higher. Interestingly, the expression profile of PPARγ 1/2 aligned with the observed differentiation potentials of MSC OC.4L.2/2.2/2.2.6 as the non-adipogenic MSC OC.4L.2.2 had the lowest expression of PPARγ 1/2 and was non-reactive to the induction media. The expression profiles of two more relevant genes, znf423 and Ebf1, also showed a correlation with the acquired adipogenic potential, as the two highly adipogenic clones MSC OC.4L.1/1.4 showed a significant increase in their expression, while the other lower adipogenic clones also showed some increase in comparison to the original MSC OC population.

By using DNA microarray to compare the non-adipogenic MSC OC and its highly adipogenic tertiary clone, MSC OC.4L.1.4, vast changes in gene expression profiles, as demonstrated in Tables 2-4, herein below. From a total of 28,815 genes in the array, 990 genes were differentially expressed (q<0.05, at least two-fold difference). 36 imprinted genes (out of 100 which are present in the array), were differentially expressed with q<0.05 (this group of genes is significantly different, p=0.0001599), 15 of them with at least two-fold difference (p=1.336E-6). Importantly, expression of the Xist RNA which is responsible for X chromosome inactivation showed an 82.87 fold reduction in the clone, implying that possibly re-activation of the X chromosome occurred, an event accompanying cell reprogramming (Table 2). In addition, the changes in imprinted gene expression were localized to specific regions in the chromosomes, suggesting that changes have occurred in imprinting control regions, which affect several imprinting genes.

TABLE 2
Significant differential expression of imprinted genes in
MSC OC and MSC OC.4L.1.4

| | | |
|---|---|---|
| Gene | Difference | q-value |

Upregulated in OC

| | | |
|---|---|---|
| Xist | 82.87 | 0.000027 |

Upregulated in OC.4.1.4

| | | | | |
|---|---|---|---|---|
| | | Asb4 | 3.39 | 0.000247 |
| Chromosome 6 | {open oversize brace} | Dlx5 | 5.09 | 0.000129 |
| | | Mest | 21.8 | 0.000121 |
| | | H19 | 20.99 | 0.000148 |
| Chromosome 7 | {open oversize brace} | | | |
| | | Igf2 | 2.8 | 0.000393 |

TABLE 3
Genes related to differentiation capacity and pluripotency are differentially
expressed between MSC OC and MSC OC.4L.1.4

| | | |
|---|---|---|
| Gene | Difference | q-value |

Upregulated in OC

| | | |
|---|---|---|
| Nestin | 12.28 | 0.000098 |
| Cebpd | 2.12726 | 0.00625 |

Upregulated in OC.4.1.4

| | | |
|---|---|---|
| lipoprotein lipase | 40.96 | 0.00004 |
| Cebpa | 13.7 | 0.00005 |
| alkaline | 10.6861 | 0.00005 |
| phosphatase | | |
| Sox9 | 3.87842 | 0.00008 |
| Pparg1 | 3.43046 | 0.00028 |

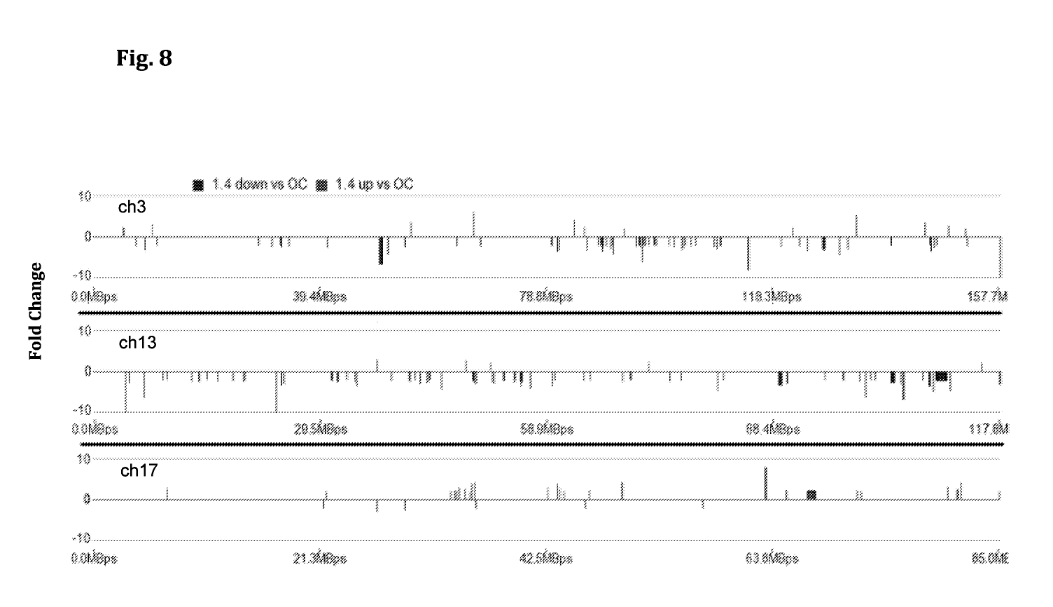

The alignment of the differentially expressed genes with their chromosomal locations, as shown in FIG. 8, demonstrate that whole chromosomes showed predominant up-regulation in gene expression in the clone (chr. 17) or in the population (chr. 3, 4, 13, 15). These findings imply that the tertiary clone under observation has undergone major epigenetic modulation. Additional gene expression changes were associated with the acquired adipogenic potential of the clone, such as, for example, elevation in lipoprotein lipase, C/EBP-alpha, PPARγ, and the like (Table 3). This was accompanied by changes genes considered to be markers of neuronal (nestin), hematopoietic (angpt1, kit1), chondrogenic (sox9), hepatocytic (hgf), and pluripotent (alp1) capacities.

As shown in Table 4, many changes in genes related to the WNT signaling pathway, which is known to be crucial for MSC fate determination.

TABLE 4
Significant differential expression of wnt related genes in MSC OC and
MSC OC.4L.1.4

| | | |
|---|---|---|
| Gene | Fold change | p-value |

Upregulated in MSC21

| | | |
|---|---|---|
| ndrg1 | 13 | 0.00068 |
| pappa2 | 9.28 | 0.00059 |
| wisp1 | 3.35 | 0.002 |
| lrp8 | 2.85 | 0.004 |
| wnt10b | 1.84 | 0.006 |
| wnt5b | 1.52 | 0.0037 |
| lrp5 | 1.5 | 0.002 |

Upregulated in MSC21.4L.1.4

| | | |
|---|---|---|
| rspo2 | 4.25 | 0.00061 |
| sfrp2 | 4.09 | 0.0004 |
| sfrp1 | 3.56 | 0.0003 |
| fzd3 | 2.35 | 0.0013 |
| wnt5a | 2.28 | 0.0016 |
| wisp2 | 2.1 | 0.003 |
| lrp4 | 1.87 | 0.0017 |
| snai2 | 1.8 | 0.001 |
| lrp11 | 1.7 | 0.04 |
| lrpap1 | 1.65 | 0.0019 |
| fzd1 | 1.59 | 0.004 |
| fzd5 | 1.45 | 0.016 |
| lef1 | 1.45 | 0.04 |

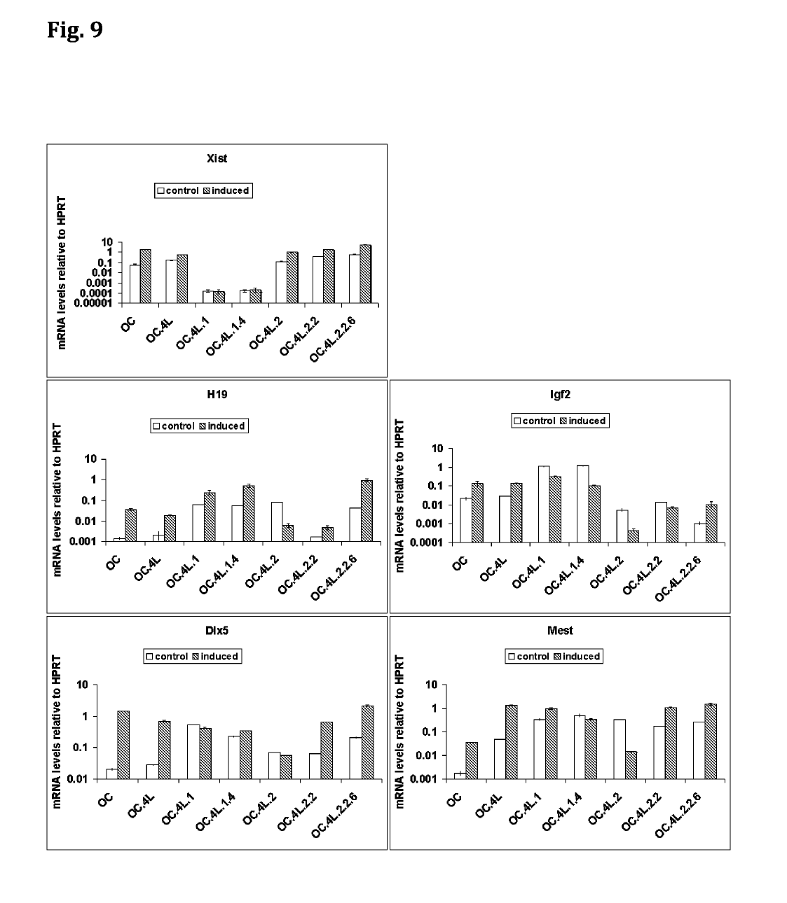

Validation of the DNA microarray results was done using real-time PCR for five different genes (H19, xist, Igf2, Dlx5 and Mest). The results are shown in the bar graphs presented in FIG. 9. MSC OC.4L.1.4 showed differences in the expression profile in comparison with MSC OC, in correlation with the microarray results. None of the genes under examination, except H19, showed strict correlation with the appearance of the adipogenic potential in other clones of the lineage. Xist expression was found to be reduced only in the highly adipogenic clones OC.4L.1/1.4, however two more adipogenic clones also had reduced expression (not shown). H19, on the other hand, had some correlation with the adipogenic potential acquisition, as the non-adipogenic clone OC.4L.2.2 had reduced expression in comparison to the adipogenic OC.4L.2 and OC.4L.2.2.6 clones. The expression of these genes was also evaluated after adipogenic induction, which resulted in the elevation of Xist, H19 and Dlx5. As can be seen, the different clones present different changes in gene expression profiles, implying that even though the cellular cloning process may result in variable gene expression profiles outcomes, all resulting in the acquisition of adipogenic potential.

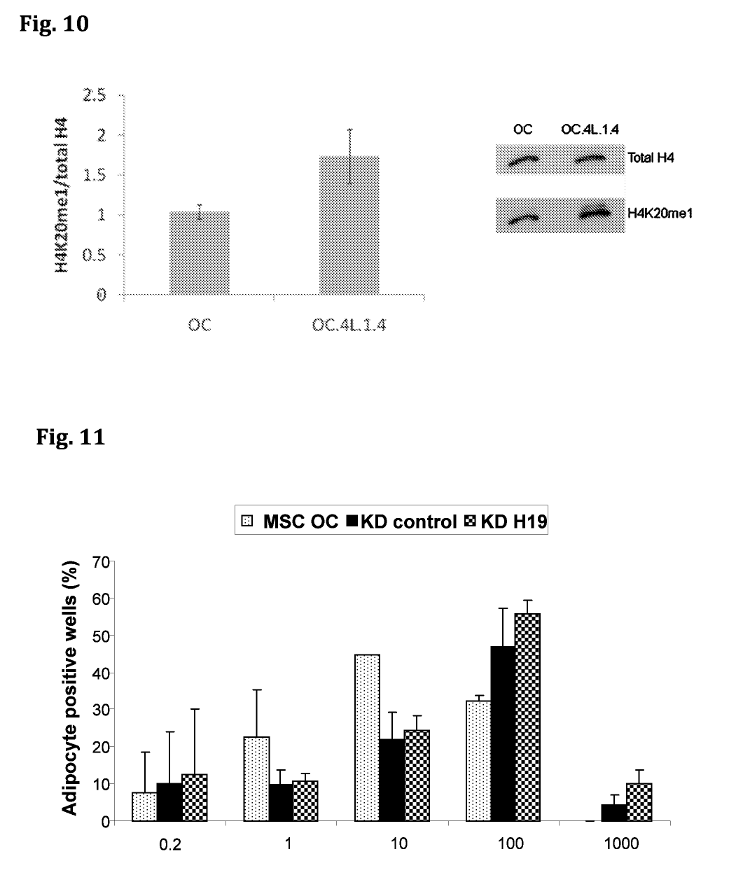

Epigenetic changes are usually associated with the modulation of the chromatin state. Three different histone methylations in MSC OC and MSC OC.4L.1.4 under standard culture conditions were compared. Global methylation amounts of H3K9me3 and H3K27me3 were not different between the two cells. Even so, there still might be differences in the methylation pattern and not with its amount in the different cells. Increased methylation in H4K20me1 was previously shown to be associated with cells undergoing adipogenesis and PPARγ expression. No significant elevation in H4K20me1 methylation after adipogenic differentiation was found in 3T3-L1 or MSC OC.4L.1.4 cells (data not shown). However, as shown in FIG. 10, H4K20me1 global methylation did show a significant up-regulation in the adipogenic clone (MSC OC.4L.1.4) compared to the non-adipogenic population (MSC OC) at non-induced culture conditions (1.7 fold increase, p=0.0267). This may imply that this methylation is indeed involved in the adipogenic process, and that MSC OC.4L.1.4 is primed towards adipogenicity by this epigenetic modulation.

### Example 6

### H19 Knock-Down Effect on Spontaneous Adipocyte Formation in Limiting Dilution

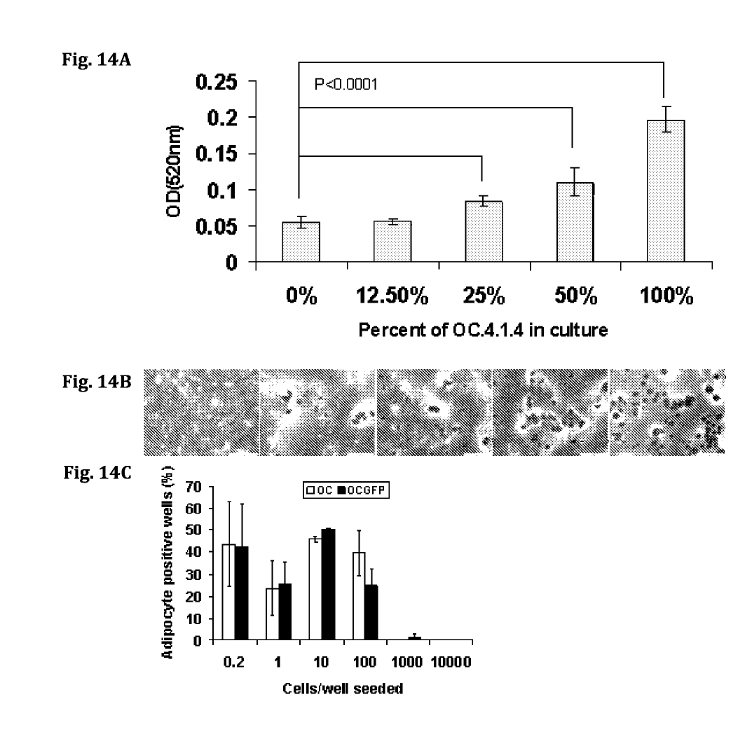

Seeding of MSC OC in dilutions in 96-well plates resulted in the spontaneous formation of adipocytes (as shown in FIGS. 11 and 14C). Seeding concentrations of up to 100 cells/well resulted in adipocytes appearance which was completely absent at higher seeding concentration. This proves that the acquisition of adipogenic potential is cell concentration dependant which does not occur when passaging cells at normal dilutions (such as 1:4). As H19 showed the best correlation with the acquisition of the adipogenic potential, this gene was knocked down (using siRNA) in MSC OC cells and its effect on spontaneous adipocyte formation in the dilution assay was evaluated. The results are presented in FIG. 11, which illustrates a bar graph of percentage of adipocyte positive wells for different cells under different experimental conditions. Untransfected MSC OC cells, control transfected MSC OC cells (i.e. cells transfected with non-specific siRNA with similar G:C content) and H19 siRNA transfected MSC OC cells were seeded at limiting dilution in 96-well plates (0.2, 1, 10, 100 and 1000 cells per well). One month after seeding (with weekly feeding), wells were inspected for adipocyte presence, and wells with one distinguishable adipocyte or more were scored positive. The percent of adipocyte positive wells from total populated wells is shown. Apparently, the siRNA transfection itself changed the appearance of spontaneous adipocytes, as both the control and the H19 knock-down resulted in a decrease of such cells in dilutions up to 10 cells/well compared to non-transfected cells.

### Example 7

### Infection Efficiency of MSC OC and MSC OA Using Lentiviral Vector

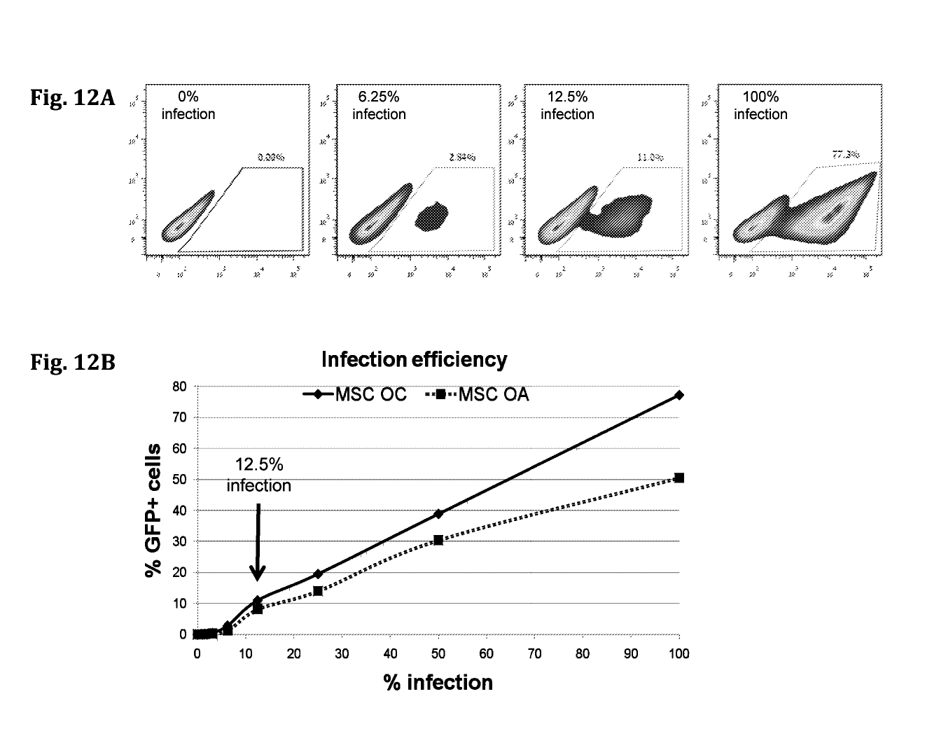

In order to demonstrate that the reprogramming events associated with cellular cloning occur on the single-cell level, lentiviral infection of GFP transgene into MSC OC was done. Lentiviruses enter dividing as well as non-dividing mammalian cells and integrate into random areas of their genome. By using multiplicity of infection which results in single viral particle infection per cell, each cell of the MSC OC population becomes uniquely labeled. To this end, a titration analysis was performed using the GFP as a marker for infection yield. The results are shown in FIG. 12, which demonstrates that both MSC OA and OC were easily infected with lentiviral particles, and when using 12.5% of the original concentration of lentiviral containing medium from transfected 293T cells, an approximately 11% infection yield was achieved.

### Example 8

### Acquisition of Adipogenic Potential Occurs on the Single Cell Level

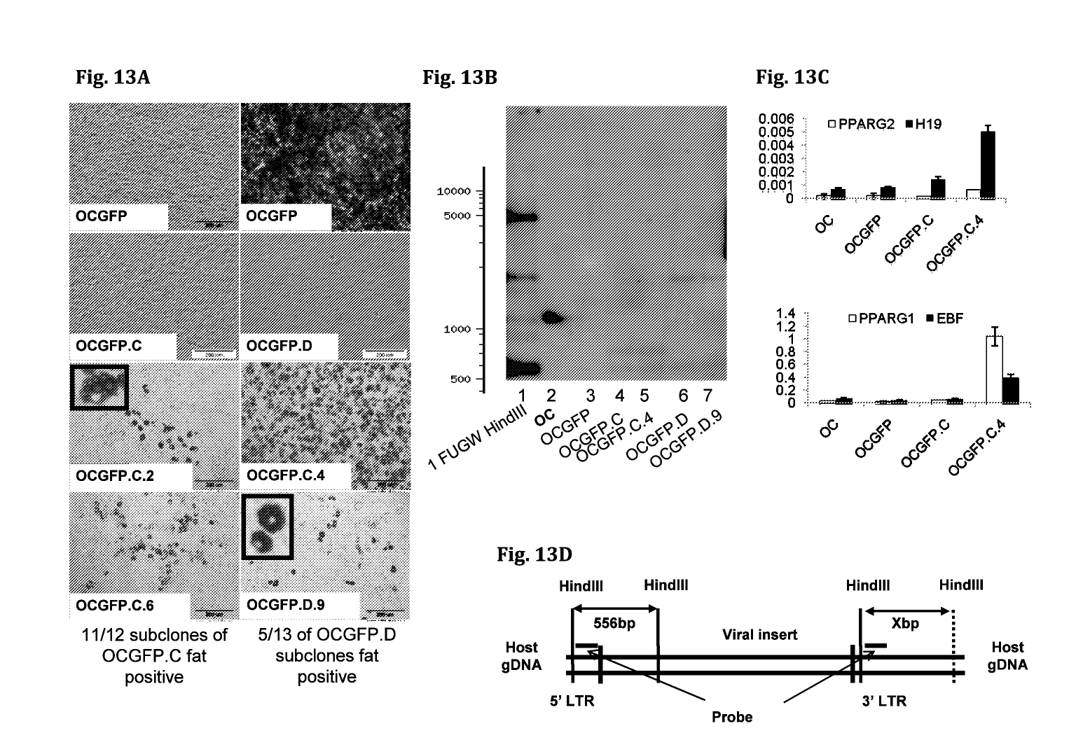

The low infected MSC OC, termed MSC OCGFP was used for the derivation of clones by seeding at a concentration of 0.2 cells/well. Single cell clones positive for GFP were selected, and two primary clones with no adipogenic potential (clones C and D) were re-cloned to obtain secondary clones. The results are shown in FIG. 13A, which demonstrates adipogenic differentiation of clonal derivations of MSC OCGFP and staining with Oil red O. These secondary clones were assayed for their adipogenic potential, and 11/12 secondary clones derived from clone C, and 5/13 clones derived from clone acquired adipogenic potential. As shown in FIG. 13B, specially designed DIG labeled probes, specific for the LTR repeats flanking the viral insert were used in a southern blot assay to detect clonal labeling. Clones C and C.4 (lanes 4 and 5, respectively, in FIG. 13B), and clones D and D.9 (lanes 6 and 7, respectively, in FIG. 13B) were analyzed and bands with lengths of ˜700 bp and 1.7 kb were visualized, respectively. Hence, a single viral integration occurred in each clone, and clones C.4 and D.9 are single cell clones derived from the primary clones C and D. As demonstrated in the illustration in FIG. 13D, as the labeled probe is specific for the flanking LTRs, a common internal control band with a length of 556 bp was detected, as expected (FIG. 13B, lowest bands in lanes 4-7). Gene expression analysis for four adipogenic genes (PPARγ 1, PPARγ 2, H19, and Ebf1) using real-time PCR, is presented in FIG. 13C. As shown in FIG. 13C, clone C.9 had elevated levels of PPARγ 1/2, H19, and Ebf1 as compared to the non-adipogenic primary clone C and the population MSC OCGFP.

### Example 9

### MSC OC does not Inhibit Adipogenic Differentiation and Acquire Adipogenic Potential in Limiting Dilution

To exclude the possibility that the adipogenic clones are present in the original MSC OC and their adipogenic differentiation is inhibited by other cells present in the population; a co-culture differentiation assay was performed. Increasing amounts of the adipogenic MSC OC.4L.1.4 (0%, 12.5%, 25%, 50% and 100%) were seeded together with MSC OC, and these co-cultures underwent adipogenic induction, as demonstrated in FIGS. 14A and 14B, which show Bar graphs of Oil red O quantification of adipogenic differentiation of MSC OC.4L.1.4 mixed with MSC OC at increasing amounts and pictographs of Oil red O staining of the cells mixtures, respectively. Even at the lowest amount of MSC OC.4L.1.4 seeded, adipocyte presence was detected, and the elevation in adipocyte numbers with increasing MSC OC.4L.1.4 concentrations was quantified. As further shown in FIG. 14C, dilutions of MSC OC and OCGFP give rise to similar yield of adipocytes, showing that the lentiviral infection of the OCGFP cells had no effect on the adipogenic process (FIG. 14C). In addition, the fact that almost 50% of the wells were positive for adipocytes when seeding 10 cells/well, dismisses the possibility that such adipogenic cells were present in the undiluted population.

### Example 10

### Gene Expression Analysis Comparing Between Dense and Dilute MSC OC Cultures

In order to delineate the basis of the cellular cloning process, DNA microarray analysis comparing between dense and dilute MSC OC cultures was performed. For dense cultures, 2.5×10⁶ cells/100 mm plate were seeded (equivalent to ˜10,000 cells/well in 96-well plate) and RNA was collected after one, two and three days in culture. Dilute cultures were seeded at 15,000 cells/100 mm plate (equivalent to ˜60 cells/well in 96-well plate) and RNA was also collected in the following consecutive 3 days. As RNA was collected on three consecutive days, the different samples of the dilute and dense cultures are not exact repeats. In accordance, only about 90 genes were differentially expressed between dense and dilute samples (q-value<0.05, fold change>2), 82 of which were over-expressed in the dense cultures. Raising the cut-off of the q-value to 0.062 gives 215 genes differentially expressed with a fold change of at least two-fold, of which, 192 are over-expressed in the dense cultures. Six possible relevant genes which were up-regulated in dense populations compared to dilute are shown in Table 5, herein below. These genes are involved in the determination of several differentiation processes, and the fact that they are reduced in dilute cultures might explain the adipogenic acquisition of these cells.

TABLE 5
Genes up-regulated in dense cultures as compared to dilute cultures

| | Fold | | |
|---|---|---|---|
| Gene | change | q-value | Relevant function |
| Cxcl5 | 55 | 0.0189 | Inhibits insulin signaling |
| Steap4 | 39 | 0.047 | Involved in insulin signaling, regulated by |
| | | | TNFα and IL-6 |
| Dio3 | 21 | 0.047 | Inactivates thyroid hormone, imprinted |
| BMP4 | 9 | 0.047 | Involved in bone and cartilage formation |
| Sfrp1 | 12.5 | 0.051 | Inhibits wnt pathway |
| Sfrp2 | 8.5 | 0.061 | Inhibits wnt pathway |

The rationale for testing RNA from the cells, obtained from consecutive days was to screen for changes in gene expression which might not occur immediately one day after seeding. Indeed, a unique pattern of gene expression correlated to increased time in dilute culture is apparent. Interestingly, one group of genes thus identified belongs to a family of small nucleolar RNA coding genes which have regulatory roles in the function and biogenesis of other small RNAs in the cells (shown in Table 6, hereinbelow). Specifically, snora44 was shown to be highly expressed one day after seeding in dilute conditions (Table 6). Two of these genes, sonrd115 and snord116 are located on chromosome 7 and are imprinted.

TABLE 6
Gene expression changes at consecutive days of dilute and dense cultures.
Values are at log-scale, and array background cut-off is 5 (below 5 = no expression)

| | Dilute culture | | | Dense culture | | |
|---|---|---|---|---|---|---|
| Gene | Day 1 | Day 2 | Day 3 | Day 1 | Day 2 | Day 3 |
| snora44 | 10.4627 | 6.35901 | 5.72363 | 6.27515 | 6.05398 | 6.21233 |
| snord118 | 5.9391 | 5.27372 | 4.35108 | 4.4235 | 3.60037 | 3.94263 |
| snord116 | 2.76967 | 5.67608 | 6.89982 | 4.65999 | 4.5165 | 3.71346 |
| snord115 | 2.69084 | 2.83907 | 5.51836 | 3.62646 | 2.70145 | 2.47136 |

When comparing the differential gene expression list of MSC OC and MSC OC.4L.1.4 to that of dilute MSC OC and dense MSC OC, some similarities can be found. While only 2 genes are up-regulated in the dilute cultures and the MSC OC.4L.1.4 clone (shown in Table 7, hereinbelow), 26 genes are down regulated in these samples compared to MSC OC (shown in Table 8 hereinbelow). For example, the presence of Pappalysin 2, a regulator of insulin-like growth factor (IGF) bioavailability, at the list, again implicates an insulin signaling pathway with the low density culture procedure.

TABLE 7
Genes over-expressed in OC.4L.1.4 and in dilute cells
compared to MSC OC

| | |
|---|---|
| OC.4L.1.4 | Dilute |

| Gene | Difference | q-value | Difference | q-value |
|---|---|---|---|---|
| Lyn | 2.0797 | 0.001139 | 3.20241 | 0.051842 |
| Mest | 21.8367 | 0.000122 | 1.98634 | 0.047992 |

TABLE 8
Genes over-expressed in MSC OC compared to
OC.4L.1.4 and dilute cells

| | OC.4L.1.4 | | Dilute | |
|---|---|---|---|---|
| Gene | Difference | q-value | Difference | q-value |
| Pappa2 | 9.28697 | 0.000129 | 25.0554 | 0.048257 |
| Cfh | 4.81262 | 0.000544 | 15.6318 | 0.052273 |
| Itga11 | 5.87353 | 7.85E−05 | 13.5027 | 0.052273 |
| Tmem45a | 4.56407 | 0.001791 | 9.00809 | 0.037371 |
| Cxcl3 | 2.15004 | 0.000441 | 8.88597 | 0.047992 |
| Kcnj2 | 3.83083 | 0.000835 | 8.78932 | 0.047992 |
| Dhrs3 | 3.75907 | 5.16E−05 | 8.55443 | 0.047992 |
| Tgfbi | 4.02251 | 5.16E−05 | 7.83982 | 0.058887 |
| Scn3a | 2.63591 | 0.000163 | 7.36167 | 0.047992 |
| Col12a1 | 2.0382 | 0.000397 | 7.15444 | 0.047992 |
| Nlrp2 | 2.6915 | 0.000388 | 5.91048 | 0.047992 |
| Bnip3 | 3.56919 | 0.000203 | 5.76629 | 0.038615 |
| Pdk1 | 3.81039 | 0.000882 | 5.02502 | 0.03814 |
| Il13ra1 | 2.73392 | 0.000347 | 4.63561 | 0.047992 |
| Pygl | 6.66669 | 7.32E−05 | 4.40906 | 0.047992 |
| Cebpd | 2.12726 | 0.006259 | 3.77152 | 0.053475 |
| Rora | 2.78938 | 0.000165 | 3.59693 | 0.047992 |
| P4ha2 | 2.59425 | 0.000129 | 3.48676 | 0.047992 |
| Slc13a5 | 2.13003 | 0.001048 | 3.33897 | 0.057795 |
| Lama2 | 2.62182 | 0.000553 | 3.32332 | 0.047992 |
| Ptch1 | 2.1986 | 0.00167 | 3.27382 | 0.051842 |
| Nos2 | 2.99272 | 0.000323 | 3.231 | 0.018914 |
| 4930583H14Rik | 4.38278 | 0.000123 | 3.21074 | 0.056005 |
| Lepr | 3.55009 | 0.000129 | 3.03279 | 0.047992 |
| Pappa | 3.32148 | 0.000158 | 2.80719 | 0.058849 |
| P4ha1 | 2.17542 | 0.000182 | 2.79886 | 0.051842 |
| Fam162a | 3.22416 | 9.43E−05 | 2.67385 | 0.052273 |
| Arrdc3 | 4.80589 | 0.000328 | 2.57906 | 0.047992 |
| Plod2 | 3.54981 | 0.00016 | 2.37637 | 0.018914 |
| Nfkbiz | 3.19619 | 0.000529 | 2.2512 | 0.057795 |
| Bcl3 | 2.21352 | 0.000386 | 2.23639 | 0.060341 |
| Lama5 | 3.41996 | 0.000174 | 2.21326 | 0.047992 |
| Slc16a2 | 2.45577 | 0.001909 | 2.1554 | 0.047992 |
| Vegfa | 2.07585 | 0.0002 | 2.09846 | 0.047992 |
| Atp2b4 | 3.30186 | 5.16E−05 | 2.07733 | 0.05267 |
| Tgfbr2 | 2.48399 | 0.000655 | 2.03077 | 0.058807 |
| Lyst | 2.59548 | 0.000163 | 2.00599 | 0.052273 |

### Example 11

### MSC OC and MSC OC.4L.1.4 have Significant Differences in the Histone Modification H4K20Me1 and as Well as Expression of Differentiation Related and Wnt Related Genes

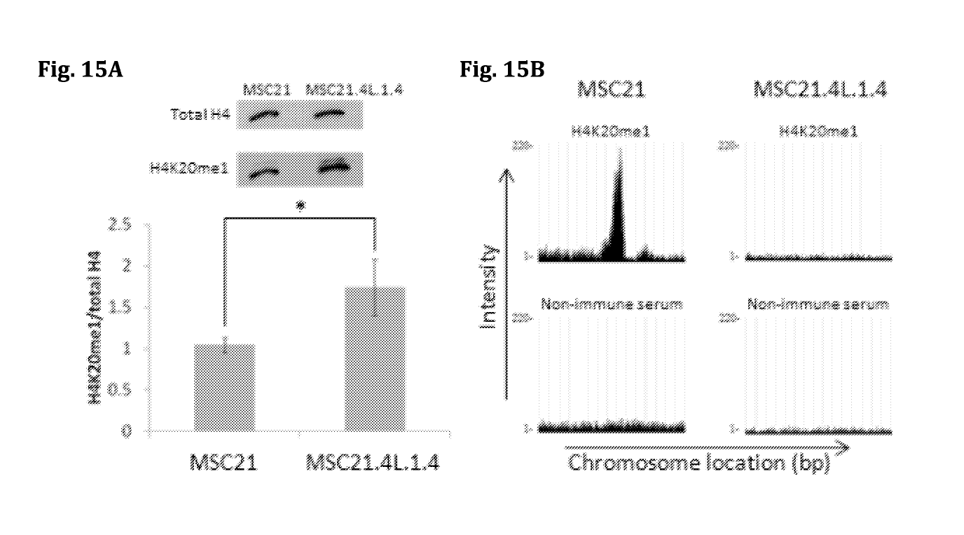

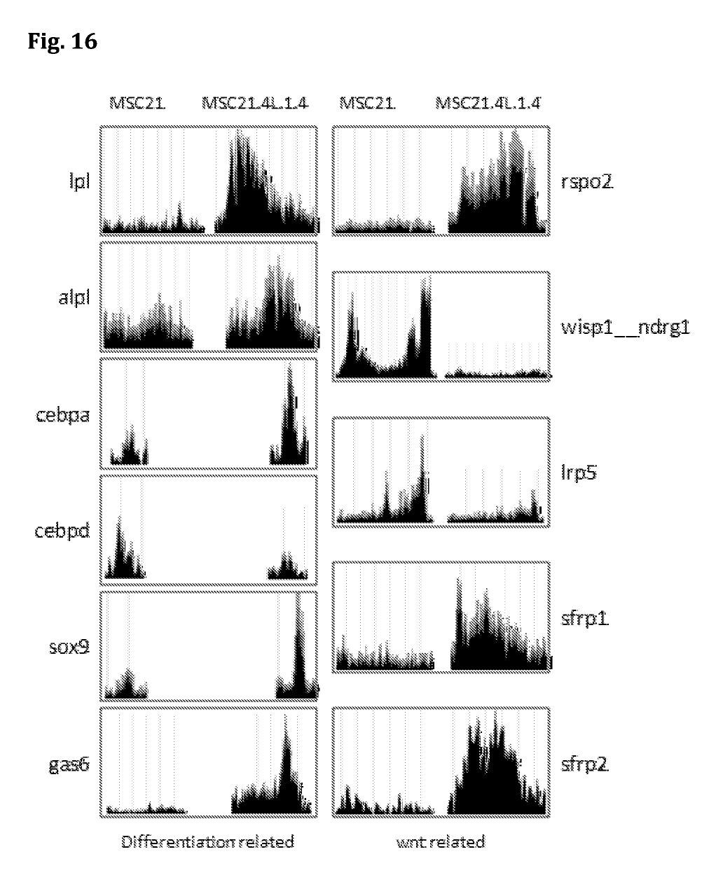

Histone extracts from confluent cells were subjected to Western Blotting using a specific H4K20me1 antibody, and densitometry relative to total H4 was calculated. Three independent histone extractions were performed (one representative blot is shown) for the calculation of methylation amount (1.7 fold increase in MSC OC.4L.1.4, p=0.0267, paired t-test). The results are presented in FIG. 15A. Methylation of H4K20me1 was identified to at the xist locus by performing chip-seq analysis in MSC OC and MSC OC.4L.1.4. For negative control, non-immune serum was used instead of the antibody. Error bars indicate mean±s.e.m. The results are presented in FIG. 15B. The results indicate that H4K20me1 is positively associated with gene expression. Specifically, this modification governs the expression of xist in MSC OC, and is absent in MSC OC.4.1.4, in which xist is not expressed. Further evaluation of the CHIP-seq results show that this modification is changed between the examined cells, and that it regulates the expression of many wnt, as well as many of the differentiation related genes which are differentially expressed in the cells. The results are shown in FIG. 16, which show bar graphs of the relative expression of the various indicated genes in the various cells.

### Example 12

### Beta-Catenin Translocate into the Nucleus in Isolated Cells

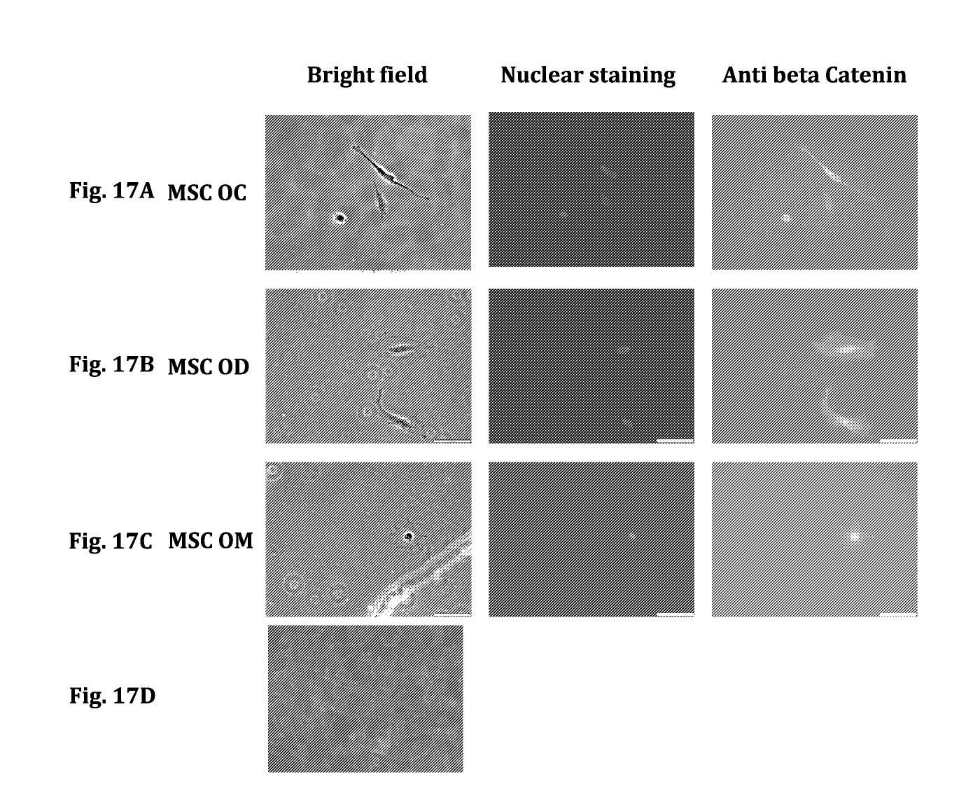

To test whether dilute cell conditions alter the canonical wnt signaling, the localization of beta-catenin in dense and dilute conditions was tested. As shows in FIGS. 17A-D, in dense conditions, beta-catenin was localized to the membrane of the cells. However, many cells in dilute conditions showed nuclear localization of beta-catenin. This phenomenon was repeated in three different MSC populations (OC, OD and OM

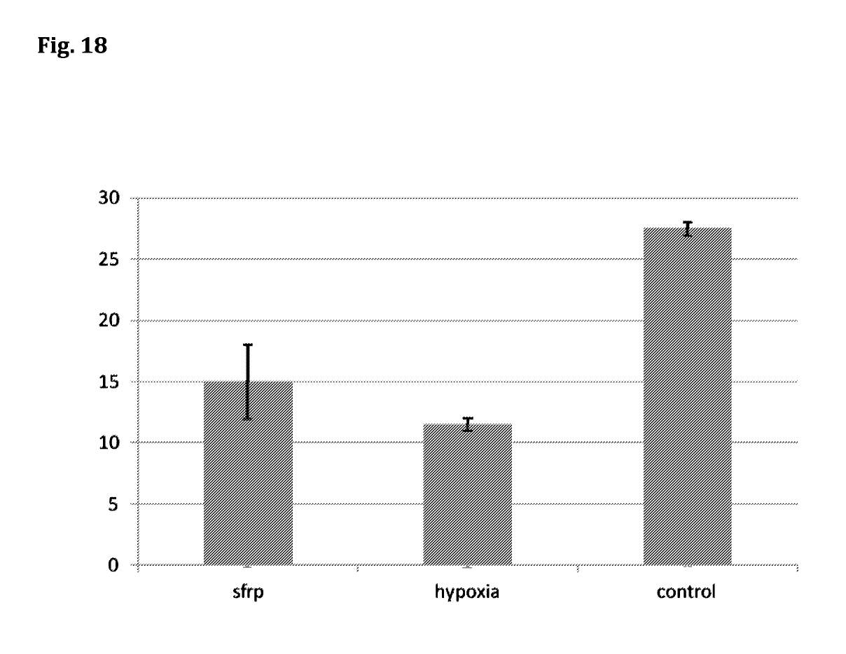

To examine the role of wnt signaling in the acquisition of the adipogenic properties of the clones, cells were seeded in 96-wells in dilute conditions, under treatment of either a wnt inhibitor (SFRP2), or under hypoxic conditions, which were previously shown to modulate this signaling cascade. The results shown in FIG. 18 (number of wells positive for adipogenic differentiation out of a 96 well plate) indicate that adipogenic acquisition is lowered in clones derived from MSC OC with wnt signaling inhibition (sfrp2, 50 ng/ml) and/or under hypoxic conditions (3% oxygen), as compared to control cells. MSC OC cells were seeded at limiting dilution of 10 cells per well in 96-well plates and treated/not treated for three weeks with sfrp2 (R&D systems) 50 ng/ml or hypoxia 3% O₂ (hypoxic incubator HERAcell, ThermoFisher).

All together, the results indicate that under wnt modulation, a decrease of about half of the amount of adipogenic clones is observed, as compared to control non-treated clonal isolates.

### Example 13

### MSCs Grown from Dilute Conditions Express Epithelial and Endothelial Markers

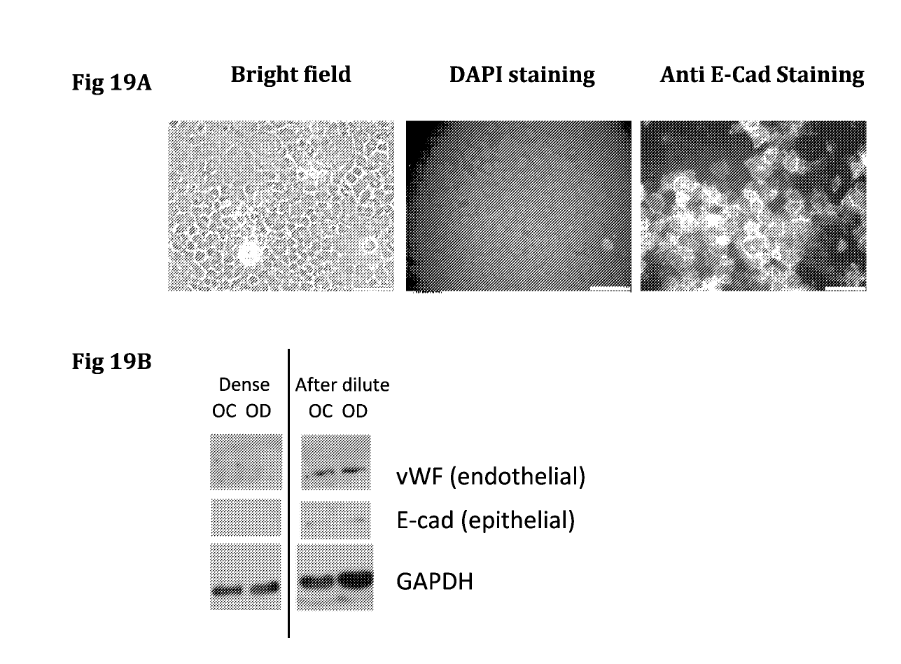

Results show that after growing cells in dilute conditions, some of the cells change their morphology and become epithelial or endothelial like cells. To examine this further, cells cultured in dilute conditions (15,000 cells in a 10 cm plate), were brought to confluence again (re-confluence) and were stained with an antibody specific to E-cadherin, a known epithelial marker. The results shown in FIG. 19A demonstrate that some of the cells were positive for this marker, while the original cell population which did not undergo culturing in dilute conditions was negative. Further protein analysis of E-cadherin, as well as an endothelial marker, vWF, was performed in two MSC populations which underwent dilute culturing. As shown in FIG. 19B, both cells showed the acquisition of expression of both markers, implying the generation of such cells in the culture due to the dilute conditions.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

1. Zipori D. Biology of stem cells and the molecular basis of the stem state. (2009) (Humana Press, Dordrecht, 2009).
2. Weigel, D and Jurgens G, 2002, Stem cells that make stems. Nature 415, 751-754
3. Brawley C and Matunis, 2004, Regeneration of male germline by spermatogonial dedifferentiation in vivo. Science, 304, 1331-1334.
4. Zipori, D. Biology of stem cells and the molecular basis of the stem state, (Humana Press, Dordrecht, 2009).
5. Zipori, D. The nature of stem cells: state rather than entity. Nat Rev Genet 5, 873-878 (2004).
6. Efroni, S., et al. Global transcription in pluripotent embryonic stem cells. Cell Stem Cell 2, 437-447 (2008).
7. Takahashi, K. and Yamanaka, S., Induction of pluripotent stem cells from mouse embryonic and adult fibroblast cultures by defined factors. Cell 126 (4), 663 (2006).
8. Maherali, N., Sridharan, R., Xie, W., Utikal, J., Eminli, S., Arnold, K., et al. (2007). Directly reprogrammed fibroblasts show global epigenetic remodeling and widespread tissue contribution. Cell Stem Cell, 1, 55-70.
9. Hochedlinger, K. & Jaenisch, R. Monoclonal mice generated by nuclear transfer from mature B and T donor cells. Nature 415, 1035-1038. (2002).
10. Conrad, S., et al. Generation of pluripotent stem cells from adult human testis. Nature 456, 344-349 (2008).
11. Kawase, E., Wong, M.D., Ding, B. C. & Xie, T. Gbb/Bmp signaling is essential for maintaining germline stem cells and for repressing bam transcription in the Drosophila testis. Development 131, 1365-1375 (2004).
12. Pevsner-Fischer M and Zipori D. (2009). Environmental signals regulating Mesenchymal progenitor cell growth and differentiation. Stem Cell Biology and Regenerative Medicine, 2009, Part II, 175-184, DOI: 10.1007/978-1-60327-227-8_{—}16.

1. A method for inducing de-differentiation of a mesenchymal stromal cell (MSC), the method comprising:
seeding or incubating the mesenchymal stromal cell (MSC) at a density of less than about 2000 cells/0.3 cm²; thereby inducing de-differentiation of the mesenchymal stromal cell (MSC).

2. The method of claim 1, wherein an exogenous gene is not expressed or introduced into the mesenchymal stromal cell (MSC).

3. The method of claim 1, wherein the de-differentiation is from an impotent differentiated mesenchymal stromal cell (nullipotent cell) to: a uni-potent cell, a bi-potent cell, a tri-potent cell or a multi-potent cell.

4. The method of claim 1, wherein the de-differentiation is from a uni-potent mesenchymal stromal cell to: a bi-potent cell, a tri-potent cell or a multi-potent cell.

5. The method of claim 1, wherein de-differentiation is from a bi-potent mesenchymal stromal cell to a tri potent cell or a multi-potent cell.

6. The method of claim 1, wherein the mesenchymal stromal cell (MSC) is de-differentiated to a cell capable of differentiating to: an osteogenic cell type, an adipogenic cell type, and/or a chondrogenic cell type.

7. The method of claim 1, wherein the density is less than about 500 cells/0.3 cm², or less than about 100 cells/0.3 cm².

8. (canceled)

9. The method of claim 1, wherein the method further comprises changing one or more growth conditions of the mesenchymal stromal cell (MSC), and wherein the one or more growth conditions are selected from the group consisting of growth media, O₇ concentration, CO₂ concentration, pressure, humidity, pH, temperature, type of substrate, and combinations thereof.

10. (canceled)

11. The method of claim 1 further comprising irradiating the cells.

12. The method of claim 1, wherein the mesenchymal stromal cell (MSC) is from human, avian or animal origin, and wherein the animal origin is murine, canine, or poultry.

13. (canceled)

14. The method of claim 12, wherein the mesenchymal stromal cell (MSC) is derived from bone marrow, adipose tissue, spleen tissue, intestine, liver tissue, muscle tissue, brain, skin, ear, bone/cartilage tissues, dental tissue, heart tissue and/or spinal cord.

15. The method of claim 1, wherein the dedifferentiated mesenchymal stromal cell (MSC) is capable of being introduced to a human or an animal.

16. A dedifferentiated mesenchymal stromal cell obtained by a method comprising a seeding or incubating a mesenchymal stromal cell (MSC) at a density of less than about 2000 cells/0.3 cm²; and
wherein an exogenous gene is not expressed or introduced into the mesenchymal stromal cell (MSC).

17. The cell of claim 16, wherein the de-differentiation is from an impotent mesenchymal stromal cell (nullipotent cell) to: a uni-potent cell, a bi-potent cell, a tri-potent cell or a multi-potent cell.

18. The cell of claim 16, wherein the de-differentiation is from a uni-potent mesenchymal stromal cell to: a bi-potent cell, a tri-potent cell or a multi-potent cell.

19. The cell of claim 16, wherein de-differentiation is from a bi-potent mesenchymal stromal cell to a tri potent cell or a multi-potent cell.

20. The cell of claim 16, wherein the mesenchymal stromal cell (MSC) is de-differentiated to cell capable of differentiating to: an osteogenic cell type, an adipogenic cell type, and/or a chondrogenic cell type.

21. The cell of claim 16, wherein the density is less than about 500 cells/0.3 cm², or less than about 100 cells/0.3 cm².

22. (canceled)

23. The cell of claim 16, wherein the method further comprises changing one or more growth conditions of the mesenchymal stromal cell (MSC).

24. The cell of claim 16, wherein the mesenchymal stromal cell (MSC) is from human or animal origin.
